(19) United States
(12) Patent Application Publication
(10) Pub. No.: US 2024/0382438 A1
(43) Pub. Date: Nov. 21, 2024

(54) Calcium Channel Blockers and Methods Thereof

(71) Applicant: University of Maryland, Eastern Shore, Princess Anne, MD (US)

(72) Inventors: Patrice L. Jackson-Ayotunde, Salisbury, MD (US); Rhashanda D. Haywood, Princess Anne, MD (US); Miguel Martin, Fruitland, MD (US); Isis Jemiyotan Amaye, Pittsburg, PA (US)

(21) Appl. No.: 18/639,921

(22) Filed: Apr. 18, 2024

Related U.S. Application Data
(60) Provisional application No. 63/496,920, filed on Apr. 18, 2023.
(60) Provisional application No. 63/496,919, filed on Apr. 18, 2023.

(51) Int. Cl. A61K 31/166

(52) U.S. Cl. CPC A61K 31/166

(57) ABSTRACT

This present disclosure is directed to use of small organic molecules calcium and sodium channel blockers as potential pharmacotherapeutics for diseases involving modulation of one or more calcium and/sodium channels (such as epilepsy).

### CROSS REFERENCE TO RELATED APPLICATIONS

This application claims priority to U.S. Application No. 63/496,920, filed Apr. 18, 2023, and U.S. Application No. 63/496,919, filed Apr. 18, 2023, the contents of which are hereby incorporated by reference in their entirety.

### FIELD OF THE INVENTION

The field of the invention relates generally to novel N-aryl enaminones and the use thereof as calcium and/or sodium channel blockers.

### DESCRIPTION

One aspect of the present disclosure pertains to discovery of small organic molecules as potential pharmacotherapeutics for diseases involving calcium channel blocking such as epilepsy. Preliminary studies performed led to the discovery of small organic molecule IAA65, a potent T-type voltage-gated calcium channel (T-VGCC) blocker. To validate the biological activity of the enaminone analogs in a battery of preclinical seizure rodent models, studies are conducted at the National Institutes of Neurological Disorders and Stroke Epilepsy Therapy Screening Program, NIH.

Prototype IAA65 (N-(5-methyl-3-oxocyclohex-1-en-1-yl)-3,5-bis(trifluoromethyl)) underwent target identification studies and was found to display significant inhibition for the T-type calcium currents. See Isis J. Amaye et al., “Evaluation of potential anticonvulsant fluorinated N-benzamide enaminones as T-type Ca²⁺ channel blockers,” Bioorg. Med. Chem. 65, 116766 (Apr. 2, 2022), which is incorporated by reference in its entirety. The meta-trifluoromethylated enaminone had effects on both the Cav3.2 and Cav3.3 subtypes. Using hit to lead optimization studies, a series of enaminone derivatives were designed based on the IAA65 template. New meta-trifluoromethylated N-benzamide enaminone compounds including RHB-56 (N-(3-oxo-5-(trifluoromethyl)cyclohex-1-en-1-yl)-3,5-bis(trifluoromethyl)benzamide), and RHB-59 (3,5-dimethyl-N-(5-methyl-3-oxocyclohex-1-en-1-yl)benzamide), are shown and described in PCT International Application No. PCT/US23/64976, entitled “BENZAMIDE ENAMINONE DERIVATIVES AND METHODS OF USE THEREOF”, filed on Mar. 27, 2023, which is incorporated by reference in its entirety.

Certain exemplary embodiments have undergone target identification studies to discover a better officious T-type calcium blocker. The fourteen trifluoromethylated N-benzamide enaminone compounds were synthesized, purified, and structure confirmed using gas chromatography/mass spectroscopy, nuclear magnetic resonance, and elemental analysis. Preliminary in vitro whole cell patch clamp experiments were performed to assess the inhibitory effects of several target analogs on voltage-activated calcium currents in human embryonic kidney (HEK-293) cells. Cav3.2 transfected HEK 293 cells were treated with 50 μM of test compounds and compared to the control group (none pretreated HEK 293 cells). The results showed that RHB-77 (N-(3-oxo-5-(trifluoromethyl)cyclohex-1-en-1-yl)-3,5-bis(trifluoromethyl)benzamide) and RHB-121 (N-(3-oxo-5-(trifluoromethyl)cyclohex-1-en-1-yl)-3-(trifluoromethoxy)benzamide) inhibited the calcium currents on T-type calcium channels 90% and 100%, respectively. The RHB-77 analog evoked a significant inhibition of T-type calcium currents in a concentration-dependent manner. Enaminone analog RHB62 caused a reduction in the Ca⁺² current, with lower potency than RHB-77. No inhibitory effect was shown at a similar concentration for analogs RHB59, RHB-95 or RHB-103.

Embodiments of the present disclosure include the synthesis of, purification, and analysis of these compounds in the electrophysiology studies on the T-type Ca²⁺ channel subunit Cav3.2. Using whole cell voltage-clamp recordings, a comparison was made of the effect of difluorinated and dimethylated N-benzamide enaminones for possible inhibitory effect on T-type Ca²⁺ channels. This can lead to potential therapeutics for several disease/disorders.

One aspect of the invention pertains to a method of treating a disease, condition, or disorder, said method comprising contacting one or more cells of a subject with one or more of the compounds of the Formula I or a pharmaceutically acceptable salt thereof:

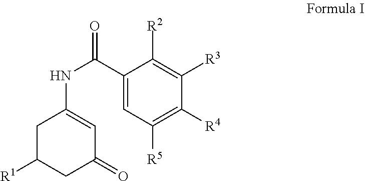

wherein
R¹ is C1-C6-alkyl (e.g. CH₃) or C1-C6-trifluoroalkyl (e.g. CF₃); and each of R², R³, R⁴, or R⁵ is independently chosen from H, halide, alkoxy, C1-C6-trifluoroalkyl (e.g. CF₃), fluorinated alkoxy (e.g. —OCF₃), fluorinated thio group (e.g. —SCF₃), CN, and NO₂.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-N displays certain chemical structures of certain embodiments of the invention. Namely, compounds FIG. 1A RHA-88, FIG. 1B RHB-14, FIG. 1C RHB-59, FIG. 1D RHB-62, FIG. 1E RHB-95, FIG. 1F RHB-77, FIG. 1G RHB-99, FIG. 1H RHB-107, FIG. 1I RHB-103, FIG. 1J RHB-123, FIG. 1K RHB-115, FIG. 1L RHB-121, FIG. 1M RHB-137, and FIG. 1N RHB-127.

FIG. 2. Organic Reaction for the Synthesis of Substituted Fluorinated N-aryl Enaminones. The amination reaction was carried out by refluxing the R₁ substituted diketone with ammonium acetate as both the amine source and base to generate the enaminone intermediate. The subsequent reaction was to generate the enaminone anion using two equivalence of sodium hydride in anhydrous tetrahydrofuran (THF). The enaminone intermediate was refluxed for about 20 minutes in sodium hydride with anhydrous THF. N-acylation with respective R₃₋₇-substituted acyl chloride gave the final N-benzamide analogs.

FIG. 3A-C Chemical Structure for Fluorinated N-Benzamide Enaminones. Namely, compounds FIG. 3A IAA65, FIG. 3B IAB15, and FIG. 3C IAA61.

FIG. 4A-E Effect of IAA61, IAB15 and IAA65 on whole-cell Ca²⁺ currents in ND7/23 cells. FIG. 4A Typical transient Ca²⁺ current in an ND7/23 cell generated by a 200 ms-voltage step to −20 mV from a holding potential of −100 mV (in this and following illustrations, the voltage protocol is presented as an insert at the lower portion of the figure). The T-type Ca²⁺ current traces generated following a 5 min perfusion with 50 mM IAA65 and drug washout are also presented. FIG. 4B Effect of 50 μM IAA65 on the T-type Ca²⁺ current densities of ND7/23 cells. Note that acute treatment of ND7/23 with 50 μM IAA65 evoked a significant reduction in the T-type Ca²⁺ current density compared with controls cells (n=10, * represents p≤0.05 vs. control, ns represents no statistical differences between the samples). Washout of IAA65 for min did not reverse the drug effect on T-type Ca²⁺ current densities. FIG. 4C Whole cell capacitance values in ND7/23 cells treated with 50 μM of IAA61, IAB15 or IAA65. Note that these enaminone analogs have no effect on cell capacitance. The number of recorded cells under each condition is presented in parenthesis above each bar. FIG. 4D Effect of 50 μM of IAA61, IAB15 or IAA65 on the densities of Ca²⁺ currents in differentiated ND7/23 cells. Note that treatment of ND7/23 cells with 50 μM of IAB15 or IAA65evoked a significant reduction in the Ca²⁺ current densities compared to non-treated cells (* represents p≤0.05 vs. vehicle). FIG. 4E Concertation-dependent effect of IAA65 on the T-type Ca²⁺ current densities generated in differentiated ND7/23 (n=9-11).

FIGS. 5A-C. Effect of IAA61, IAB15 and IAA65 on whole-cell Na⁺ currents in differentiated ND7/23 cells. FIG. 5A Typical transient Na⁺ current in an ND7/23 cell generated by a 20 ms-voltage step to −10 mV from a holding potential of −100 mV. FIG. 5B Whole cell capacitance values in ND7/23 cells treated with 50 μM of IAA61, IAB15 or IAA65. Note that these enaminone analogs have no effect on cell capacitance. The number of recorded cells under each condition is presented in parenthesis above each bar. FIG. 5C Effect of 50 μM of IAA61, IAB15 or IAA65 on the densities of Na⁺ currents in differentiated ND7/23 cells. Note that treatment of ND7/23 cells with 50 μM of IAB15 or IAA65 has no effect on the Na⁺ current densities compared to non-treated cells.

FIGS. 6A-B. Effect of IAC41, IAB65, or TTA40 on whole-cell Ca²⁺ currents in differentiated ND7/23 cells. FIG. 6A Whole cell capacitance values in ND7/23 cells treated with 50 μM of IAC41, IAB65, or TTA40. Note that these enaminone analogs have no effect on cell capacitance. The number of recorded cells under each condition is presented in parenthesis above each bar. FIG. 6B Effect of 50 μM of IAC41, IAB65, or TTA40 on the densities of Ca²⁺ currents in differentiated ND7/23 cells. Note that treatment of ND7/23 cells with 50 μM of IAC41, IAB65, or TTA40 has no effect on Ca²⁺ current densities compared to non-treated cells.

FIGS. 7A-D Effect of IAA65 on whole-cell Ca²⁺ currents in Cav3.2-stable transfected HEK 293 cells. FIG. 7A Transient T-type Ca²⁺ currents in a Cav3.2-stable transfected HEK 293 cells generated by 200 ms-voltage steps to various potentials from a holding potential of −100 mV. Typical currents generated in control and IAA65 (10 μM)-treated cells. FIG. 7B Current-voltage relationship in control (filled circles, n=10) and IAA65 (10 μM, filled squares, n=12)-treated cells. Note that peak currents are generated at voltage steps of between −30 to −20 mV. There is no significant shift in the voltage-dependence of T-type Ca²⁺ currents following exposure to 10 μM IAA65. FIG. 7C Concentration-dependent effect of IAA65 (1-50 PM) the cell densities of Cav3.2-stable transfected HEK 293 cells. Note that treatment of Cav3.2-stable transfected HEK 293 cells with increasing concentrations of IAA65 has no effect on cell capacitance. The number of recorded cells under each condition is presented in parenthesis above each bar. FIG. 7D Effect of IAB65 (1-50 μM) on the densities of Ca²⁺ currents in Cav3.2-stable transfected HEK 293. Note that treatment of Cav3.2-stable transfected HEK 293 increasing concentrations of IAA65 evokes a significant reduction in T-type Ca²⁺ current densities compared to non-treated cells.

FIGS. 8A-F. Effect of IAA65 on activation and inactivation time constants (τ), steady-state inactivation, and time dependence of recovery from short-term inactivation in Cav3.2-stable transfected HEK 293 cells. FIG. 8A-B IAA65 (10 μM) has no effect on the activation and inactivation time constants (τ), respectively. Filled circles represent the voltage-dependent activation or inactivation T in control cells (n=13), whereas filled squares represent the activation or inactivation τ of IAA65 (10 μM, n=13)-treated cells. FIG. 8C. Representative family of current traces used to study steady-state inactivation. Current traces were generated by applying a 1000 ms pre-pulse between −100 and −30 mV, followed by a test pulse to −20 mV for 200 ms. FIG. 8D. Voltage dependence of steady-state inactivation in control (filled circles, n=14) and IAA65 treated cells (filled squares, n=14). The solid line represents the best fit obtained with a Boltzmann equation for steady-state inactivation. FIG. 8E. Representative family of current traces used to assess time dependence of recovery from short-term inactivation. The time dependence of recovery from short-term inactivation was determined by applying a 200 ms pre-pulse to +30 mV from a holding potential of −100 mV, following an identical test potential, but with increasing duration of the inter-pulse interval (Δt=0-1500 ms). Note that increasing the duration of the inter-pulse interval (Δt) results in the recovery of the peak current. FIG. 8F. Plot of the normalized peak current as a function of the duration of the inter-pulse potential in control (filled circles, n=9) and IAA65 treated cells (filled squares, n=9). Note that treatment of Cav3.2-stable transfected HEK 293 cells with IAA65 (10 μM) evokes a rightward shift in the recovery time of steady-state inactivation.

FIGS. 9A-D Effect of IAA65 on whole-cell Ca²⁺ currents in Cav3.3-stable transfected HEK 293 cells. FIG. 9A. Representative family of transient T-type Ca²⁺ currents in a Cav3.3-stable transfected HEK 293 cells generated by 500 ms-voltage steps to various potentials from a holding potential of −100 mV. Typical traces represent recordings from control and IAA65 (10 μM)-treated cells. FIG. 9B. Current-voltage relationship in control (filled circles, n=8) and IAA65 (10 μM, filled squares, n=11)-treated cells. Note that peak currents are generated at voltage steps of between −40 to −30 mV. There is no significant shift in the voltage-dependence of T-type Ca²⁺ currents following exposure to 10 μM IAA65. FIG. 9C. Concentration-dependent effect of IAA65 (1-50 μM) the cell densities of Cav3.2-stable transfected HEK 293 cells. Note that treatment of Cav3.3-stable transfected HEK 293 cells with increasing concentrations of IAA65 has no effect on cell capacitance. The number of recorded cells under each condition is presented in parenthesis above each bar. FIG. 9D. Effect of IAB65 (1-50 μM) on the densities of Ca²⁺ currents in Cav3.3-stable transfected HEK 293. Note that treatment of Cav3.3-stable transfected HEK 293 increasing concentrations of IAA65 evokes a significant reduction in T-type Ca²⁺ current densities compared to non-treated cells.

FIGS. 10A-D. Effect of IAA65 on activation and inactivation time constants (τ), steady-state inactivation, and time dependence of recovery from short-term inactivation in Cav3.3-stable transfected HEK 293 cells. FIG. 10A-B. Effect of 10 μM IAA65 on the activation and inactivation time constants (τ), respectively. Filled circles represent the voltage-dependent activation or inactivation τ in control cells (n=11), whereas filled squares represent the activation or inactivation τ of IAA65 (10 μM, n=10)-treated cells. Note that 10 μM IAA65 evokes a significant increase in the activation time constants (τ) and a significant reduction in the inactivation time constants (τ) of T-type Ca²⁺ currents in Cav3.3-stable transfected HEK 293 cells. FIG. 10C. Representative family of current traces used to study steady-state inactivation. Current traces were generated by applying a 1000 ms pre-pulse between −100 and −30 mV, followed by a test pulse to −20 mV for 500 ms. FIG. 10D. Voltage dependence of steady-state inactivation in control (filled circles, n=8) and IAA65 treated cells (filled squares, n=9). The solid line represents the best fit obtained with a Boltzmann equation for steady-state inactivation. Note that IAA65 (10 μM) has no effect on the steady-state inactivation of T-type Ca²⁺ currents in Cav3.3-stable transfected HEK 293 cells.

FIGS. 11A-D. Effect of RHB62, RHB95, RHB121, RHB107, and ABA58 on T-type Ca²⁺ currents in Cav3.2 stable transfected HEK 293 cells. FIG. 11A. Typical transient Ca²⁺ current in an HEK 293-Cav3.2 cell generated by a 200 ms-voltage step to −20 mV from a holding potential of −100 mV (in this and following illustrations, the voltage protocol is presented as an insert at the lower portion of the figure). The T-type Ca²⁺ current traces generated following a 5 min perfusion with 50 mM RHB121. FIG. 11B. Whole cell capacitance values in HEK 293-Cav3.2 cells treated with 50 μM of RHB62, RHB95, RHB121, RHB107, and ABA58. Note that these enaminone analogs have no effect on cell capacitance. The number of recorded cells under each condition is presented in parenthesis above each bar. FIG. 11C. Effect of 50 μM of RHB62, RHB95, RHB121, RHB107, and ABA58 on the densities of T-type Ca²⁺ currents in HEK 293-Cav3.2 cells. Note that treatment of HEK 293-Cav3.2 cells with 50 μM of RHB62, RHB95, and RHB121 evoked a significant reduction in the Ca²⁺ current densities compared to non-treated cells (* represents p≤0.05 vs. vehicle). FIG. 11D. Concertation-dependent effect of RHB 121 on the T-type Ca2+ current densities generated in HEK 293-Cav3.2 (n=9-11).

FIGS. 12A-D. Effect of RHB121 on the activation and inactivation time constants (τ) and steady-state inactivation in Cav3.2-stable transfected HEK 293 cells. FIG. 12A-B. RHB121 (10 μM) has no effect on the activation time constants (τ), whereas the effect on the inactivation time constants (τ) was noticeable at voltages between −10 and =10 mV, respectively. Filled circles represent the voltage-dependent activation or inactivation τ in control cells, whereas filled squares represent the activation or inactivation τ in RHB121 (10 μM)-treated cells. FIG. 12C. Voltage dependence of steady-state inactivation in control (filled circles) and RHB121-treated cells (filled squares). The solid line represents the best fit obtained with a Boltzmann equation for steady-state inactivation. FIG. 12D. Differences in the V1/2 and κ values obtained from the fitting of the steady-state inactivation traces in control and RHB121-treated cells, following the best fit obtained with a Boltzmann equation (* represents p≤0.05 vs. control non-treated cells).

FIGS. 13A-C. Effect of RHB121 on whole-cell Na⁺ currents in differentiated ND7/23 cells. FIG. 13A. Typical transient Na⁺ current in an ND7/23 cell generated by a 20 ms-voltage step to −10 mV from a holding potential of −100 mV. FIG. 13B. Whole cell capacitance values in differentiated ND7/23 cells treated with 50 μM of RHB 111. Note that this enaminone analog has no effect on cell capacitance. The number of recorded cells under each condition is presented in parenthesis above each bar. FIG. 13C. Effect of 50 μM of RHB121 on the densities of Na⁺ currents in differentiated ND7/23 cells. Note that treatment of ND7/23 cells with 50 μM of RHB121 has no effect on the Na⁺ current densities compared to non-treated cells (ns indicates nonsignificant differences between control and RHB121-treated cells).

FIGS. 14A-C. Effect of RHB121 on the intracellular calcium signals generated in differentiated ND7/23 cells following stimulation with 30 mM extracellular K+ ions or the calcium ionophore ionomycin (10 μM). FIG. 14A. Note that treatment of differentiated ND7/23 cells with 30 mM of KCl or Ionomycin generates a significant increase in intracellular calcium. FIG. 14B. In differentiated ND7/23 cells pre-treated with 50 μM RHB121, there is a significant reduction in the intracellular calcium signal generated by stimulation with 30 mM KCl or Ionomycin. FIG. 14C. Relative changes in the intracellular calcium signal generated in differentiated ND7/23 cells by 30 mM KCl or Ionomycin (10 μM) in control or RHB121-treated cells. Control groups were arbitrarily assigned a 100% intracellular calcium signal.

FIG. 15. Effect of RHB121, RHB62, and RHB95 on the cell viability of LNCaP prostate cancer cells. Note that treatment of LNCaP cells with 50 μM of RHB121, RHB62, or RHB95 for 48 hours has no effect on the cell viability as assessed by the XTT assay. Cell viability was expressed as a percent of control LNCaP cells that received no treatment. Control groups were arbitrarily assigned a 100% viability value.

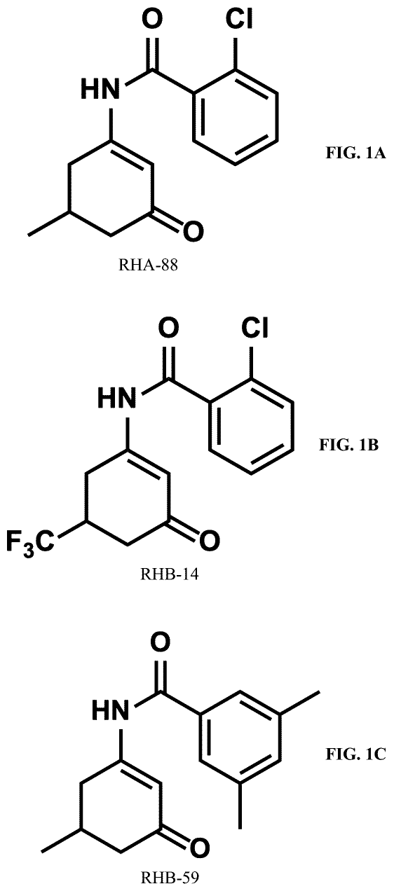

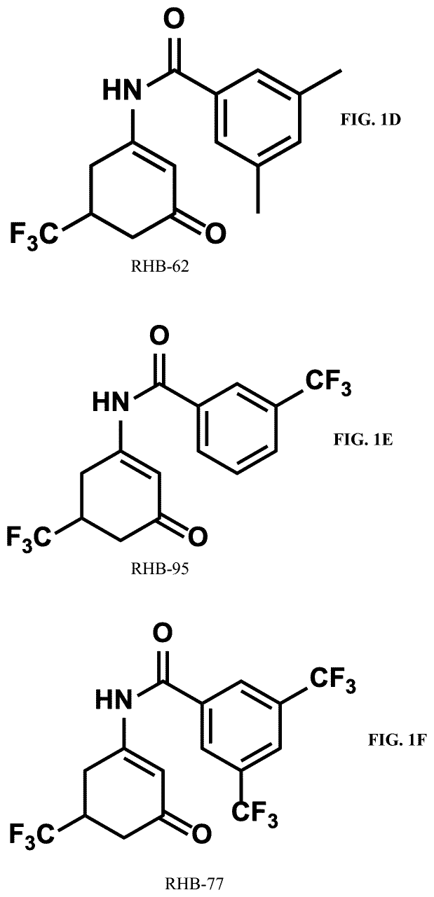

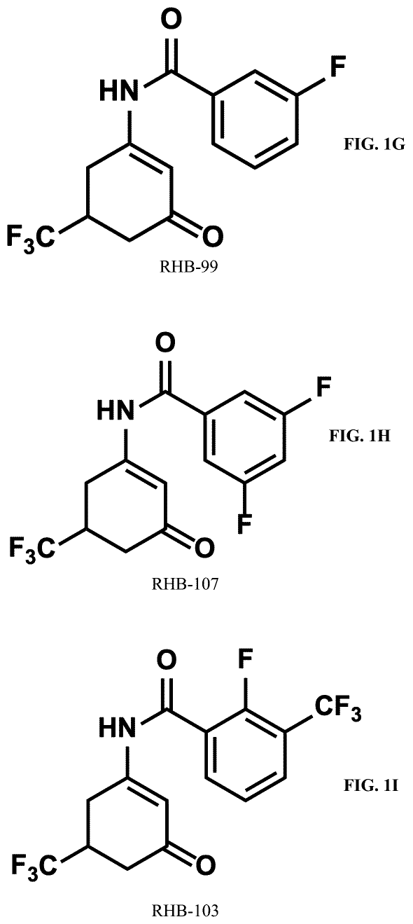

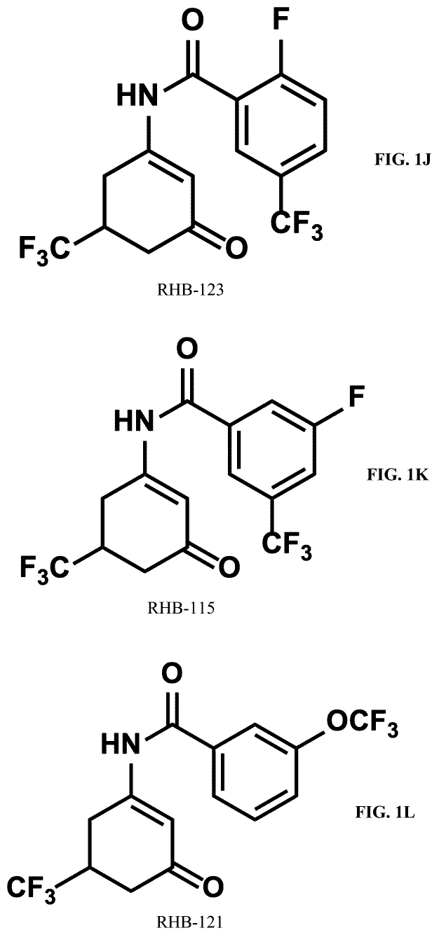

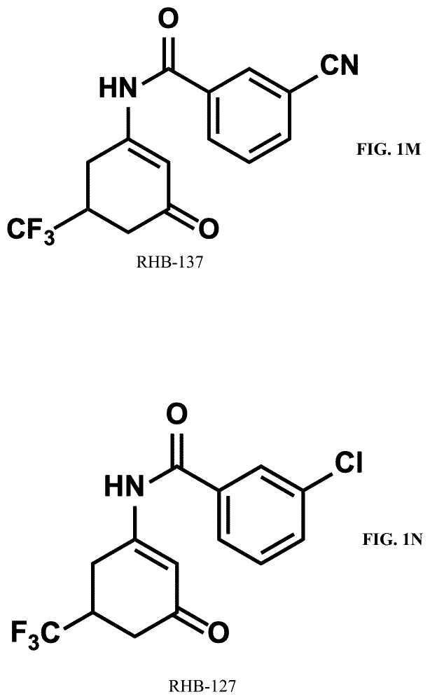

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”.

As used herein, the term “about” refers to a ±10% variation from the nominal value. It is to be understood that such a variation is always included in any given value provided herein, whether or not it is specifically referred to.

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Moreover, all ranges disclosed herein are to be understood to encompass any and all subranges (including all fractional and whole values) subsumed therein.

As used herein, the term “alkyl” or “optionally substituted alkyl” refers to C₁-C₆ unsubstituted alkyl or alkyl having one or more substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkyl aminocarbonyl, dialkylamino-carbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g. C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms. The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonylosy, aryl carbonyloxy, alkoxycarbonyloxy, aryloxycarbonyloxy, carboxylate, alkylcarbonyl, arylcarbonyl, alkoxycarbonyl, aminocarbonyl, alkyl aminocarbonyl, dialkylaminocarbonyl, alkylihiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, aryltio, thiocarboxylate, sulfates, alkylsulfinyl, sulfonato. sulfamoyl, sulfonamido, nitro, trifluoromethyl, cyano, heterocyclyl, alkylaryl, or an aromatic or heteroaromatic moiety. Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahy dropyridinyl.

As used herein, “amine” or “amino” refers to unsubstituted or substituted —NH.sub.2. “Alkylamino” includes groups of compounds wherein nitrogen of —NH.sub. 2 is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties. respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. “Analog” “analogue, and “derivative” are used herein interchangeably and refer to a compound that possesses the same core as the parent compound but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

As used herein, the term “aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc. Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine. In the case of multicyclic aromatic rings, only one of the rings needs to be aromatic e.g., 2,3-dihydroindole), although all of the rings may be aromatic (e.g. quinoline). The second ring can also be fused or bridged. The aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkyl carbonyloxy, aryl carbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato. sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

As used herein, the term “arylalkyl” or an “aralkyl” moiety is an alkyl substituted with an aryl (e.g. phenylmethyl (benzyl)). An “alkylaryl” moiety is an aryl substituted with an alkyl (e.g., methylphenyl).

As used herein, the term “fluorinated thio group” refers to the following chemical moiety: —S-alkyl wherein said alkyl suis substituted by one more fluorine atoms, for example —SCF₃, SCHF₂, etc.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

As used herein, the term “carboxyl” refers to —COOH or its C₁-C₆ alkyl ester.

As used herein, the phrase “effective amount” or “therapeutically effective amount” of a compound or pharmaceutical composition refers to an amount sufficient to achieve the intended purpose, for example, preventing or reducing the number of seizures in a mammal, especially a human, including without limitation decreasing number or intensity of a seizure or preventing occurrence or duration of a seizure in an animal prior to administration, i.e., prophylactic administration. The terms also refer to an amount of a compound or salt thereof or composition thereof to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. In a preferred aspect, the disease or condition to be treated is a seizure or a seizure disorder.

The term “ester” includes compounds or moieties which contain a carbon, or a heteroatom bound to an oxygen atom which is bonded to the carbon of a carbonyl group.

The term “ester” includes alkoxycarboxy groups such as methoxycarbonyl, ethoxycarbonyl, propoxycarbonyl, butoxycarbonyl, pentoxycarbonyl, etc.

As used herein, “excipient” is a substance, other than the active drug substance, e.g. gaboxadol, of a pharmaceutical composition, which has been appropriately evaluated for safety and are included in a drug delivery system to either aid the processing of the drug delivery system during its manufacture; protect; support; enhance stability, bioavailability, or patient acceptability; assist in product identification; or enhance any other attributes of the overall safety and effectiveness of the drug delivery system during storage or use.

As used herein, “halo” or “halogen” refers to fluoro, chloro, bromo and iodo. The term “haloalkyl” or “haloalkoxyl” refers to an alkyl or alkoxyl substituted with one or more halogen atoms.

As used herein, the term “heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e. g., 1 or 1-2 or 1-3 or 1-4 or 1-5 or 1-6 heteroatoms, or e.g., 1, 2, 3, 4, 5 or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N->0 and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole. triazole, tetrazole, pyrazole, oxazole, isoxazole, pyridine, pyrazine, pyridazine, pyrimidine, and the like.

The term “hydroxy” or “hydroxyl” includes groups with an —OH.

As used herein, the term “mitigate” or “mitigation” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as a seizure is alleviated if the severity or frequency of the seizure is reduced.

As used herein, “pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As used herein, “dosage” is intended to encompass a formulation expressed in terms of μg/kg/day, μg/kg/hr, mg/kg/day or mg/kg/hr. The dosage is the amount of an ingredient administered in accordance with a particular dosage regimen. A “dose” is an amount of an agent administered to a mammal in a unit volume or mass, e.g., an absolute unit dose expressed in mg or ug of the agent. The dose depends on the concentration of the agent in the formulation, e.g., in moles per liter (M), mas per volume (m/v), or mas per mas (m/m). The two terms are closely related, as a particular dosage results from the regimen of administration of a dose or doses of the formulation. The particular meaning in any case will be apparent from context.

As used herein, the term “seizures” and related “seizure disorders” that can treated, prevented or mitigated by administering a compound of the invention include, but are not limited to, epilepsy and related disorders and their attendant seizure symptoms. Non-limiting examples of seizure disorders include, but are not limited to, epilepsy (including but not limited to, localization-related epilepsies, generalized epilepsies, epilepsies with both generalized and/or local seizures, and the like), seizures associated with Lennox-Gastaut syndrome, seizures as a complication of a disease or condition (such as seizures associated with encephalopathy, phenylketonuria, juvenile Gaucher's disease, Unvericht-Lundborg's progressive myoclonic epilepsy, stroke, head trauma, stress, hormonal changes, drug use or withdrawal, alcohol use or withdrawal, sleep deprivation, fever, infection, brain cancer, essential tremor syndrome and restless limb syndrome, and the like), and the like. In embodiments, the disorder is selected from epilepsy (regardless of type, underlying cause or origin), essential tremor syndrome, or restless limb syndrome. In embodiments, the seizure disorder is a disease or condition that is mediated by elevated persistent sodium current and/or other neural ionotropic abnormalities. As will be recognized in the art, a characteristic that distinguishes categories of seizures is whether the seizure activity is partial (e.g., focal) or generalized. In an embodiment, a compound/composition of the present disclosure is used to treat partial and/or generalized seizures. Partial seizures are considered those in which the seizure activity is restricted to discrete areas of the cerebral cortex. As is known in the art, if consciousness is fully preserved during the seizure, the seizure is considered to be a simple-partial seizure. If consciousness is impaired, the seizure is considered to be a complex-partial seizure. Within these types of seizures are included those that initiate as partial seizures and subsequently extend through the cortex; these are considered partial seizures with secondary generalization. Generalized seizures encompass distant regions of the brain simultaneously in a bilaterally symmetric manner and can include sudden, brief lapses of consciousness, such as in the case of absence or petit mal seizures, without loss of postural control. Atypical absence seizures usually include a longer period of lapse of consciousness, and more gradual onset and termination. Generalized tonic-clonic or grand mal seizures, which are considered to be the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 seconds, the tonic phase of the seizure typically evolves into the clonic phase, produced by the superimposition of periods of muscle relaxation on the tonic muscle contraction. The periods of relaxation progressively increase until the end of the ictal phase, which usually lasts no more than one minute. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 seconds. Consciousness is briefly impaired, but there is usually no postictal confusion. Myoclonic seizures are characterized by a sudden and brief muscle contraction that may involve one part of the body or the entire body. It is considered that the present disclosure is applicable for prophylaxis and/or therapy of any of the foregoing types of seizures, which are described for illustration but are not meant to be limiting. In embodiments, the disclosure is pertinent to treatment of epilepsy. In embodiments, the epilepsy is selected from idiopathic, cryptogenic, symptomatic, general and focal epilepsy. In embodiments, the disclosure is pertinent to treatment of pharmacoresistant epilepsy. As used herein, the term pharmacoresistant epilepsy means an epilepsy that is not controlled despite use of at least two drugs that are suitable for the type of epilepsy and have been appropriately prescribed at maximum tolerated doses. In embodiments the pharmacoresistant epilepsy is one where three such drugs trials have failed to eliminate the seizures. Those skilled in the art will recognize that the chances of controlling epilepsy decline sharply after failure of the second or third antiepileptic drug trial, and thus the present disclosure provides an approach designed to address these failed treatment cases.

As used herein, the term “treating” or “treatment” refers to alleviating, attenuating or delaying the appearance of clinical symptoms of a disease or condition in a subject that may be afflicted with or predisposed to the disease or condition, but does not yet experience or display clinical or subclinical symptoms of the disease or condition. In certain embodiments, treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” may also refer to inhibiting the disease or condition, e.g., arresting or reducing its epileptic or at least one clinical or subclinical symptom thereof “Treating” or “treatment” further refers to relieving the disease or condition, e.g. causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic treatment are two separate embodiments of the disclosure herein. The inhibition is a measurable inhibition compared to a suitable control. In one embodiment, inhibition is at least 10 percent inhibition compared to a suitable control. That is, the rate of enzymatic activity or the amount of product with the inhibitor is less than or equal to 90 percent of the corresponding rate or amount made without the inhibitor. In various other embodiments, inhibition is at least 20, 25, 30, 40, 50, 60, 70, 75, 80, 90, or 95 percent inhibition compared to a suitable control. In one embodiment, inhibition is at least 99 percent inhibition compared to a suitable control. That is, the rate of enzymatic activity or the amount of product with the inhibitor is less than or equal to I percent of the corresponding rate or amount made without the inhibitor.

The terms “multi-substituted” “di-substituted” and “mono-substituted” refer to the number of substituents on a benzene ring. A molecule with more than 2 substituents on the benzene ring is “multi-substituted”, a molecule with 2 substituents on the benzene ring is “di-substituted”, and a molecule with 1 substituent on the benzene ring is “mono-substituted”.

The terms “ortho”, “meta” and “para” substituted refers to the position of the substituent on a benzene ring. Ortho-substitution occurs when two substituents on a benzene ring are in the 1,2-position, or next to each other on the benzene ring. Meta-substituted occurs when two substituents on a benzene ring are in the 1,3-position or separated by one carbon in the benzene ring. Para-substitution occurs when two substituents on a benzene ring are in the 1,4-position or separated by two carbons in the benzene ring.

### LIST OF EMBODIMENTS

1. A method of treating a disease, condition, or disorder, said method comprising contacting one or more cells of a subject with one or more of the compounds of the Formula I or a pharmaceutically acceptable salt thereof:

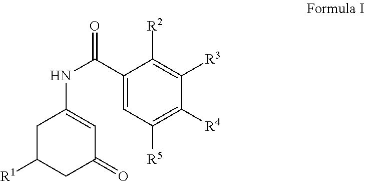

wherein
R¹ is C₁-C₆-alkyl (e.g., CH₃) or C₁-C₆-trifluoroalkyl (e.g. CF₃); and
each of R², R³, R⁴, and R⁵ is independently chosen from H, halide, alkoxy, C₁-C₆-trifluoroalkyl (e.g. CF₃), fluorinated alkoxy (e.g. —OCF₃), fluorinated thio group (e.g. —SCF₃), CN, and NO₂.

2. The method of embodiment 1, wherein said compound of Formula I is:

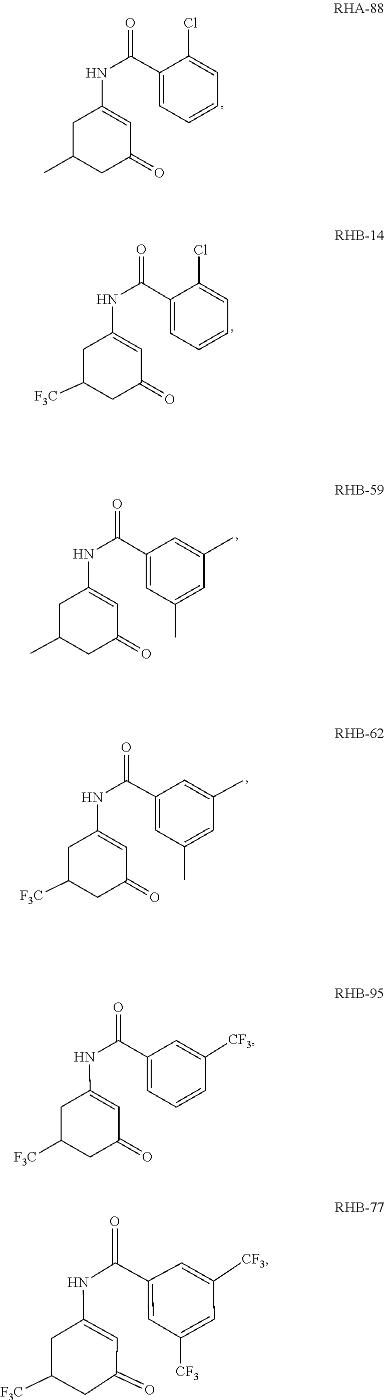

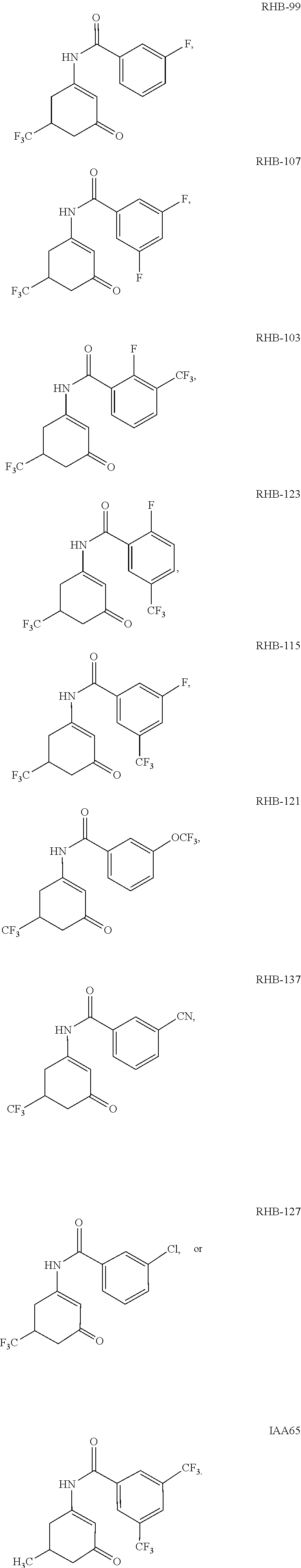

3. The method of embodiment 1, wherein said compound of Formula I is:

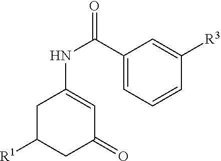

Formula Ia
wherein each of R², R⁴, and R⁵ is H.

4. The method of any of the preceding embodiments, wherein said disease, condition and/or disorder involves the modulating (e.g. inhibition) of one or more calcium channels and/or one or more sodium channels.

5. The method of any of the preceding embodiments, wherein said calcium channel is T-type.

6. The method of any of the preceding embodiments, wherein said calcium channel is Cav3.2 and/or Cav3.3 subtype.

7. The method of any of the preceding embodiments, wherein said disease, condition and/or disorder is a neurological disease or disorder, pain disease or disorder, sleep disease or disorder, and/or anxiety disease or disorder.

8. The method of any of the preceding embodiments, wherein said disease or disorder is seizure disorder.

9. The method of embodiment 2, wherein the compound is chosen from:

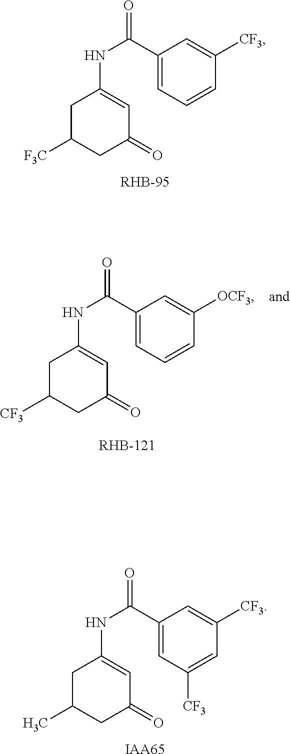

10. The method of embodiment 9, wherein the compound is:

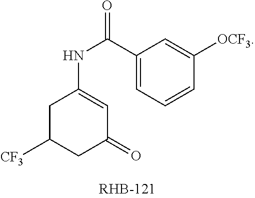 [SMILES: O=C1C=C(NC(=O)c2cccc(OC(F)(F)F)c2)CC(C(F)(F)F)C1]

11. The method of embodiment 9, wherein the compound is:

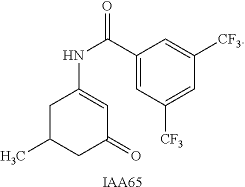

12. The method of embodiment 1, wherein each of R¹ and R³ is C₁-C₆-trifluoroalkyl.

13. The method of embodiment 1, wherein R¹ is C₁-C₆-trifluoroalkyl.

14. The method of embodiment 1, wherein each of R³ is C₁-C₆-trifluoroalkyl.

15. The method of embodiment 12, wherein each of R¹ and R³ is —CF₃.

16. The method of embodiment 1, wherein R¹ is C₁-C₆-trifluoroalkyl and R³ is fluorinated alkoxy.

17. The method of embodiment 16, wherein R¹ is —CF₃ and R³ is —OCF₃.

18. The method of embodiment 1, wherein each of R³ is —OCF₃.

### Examples

The following examples are provided solely to illustrate the present invention and are not intended to limit the scope of the invention, described herein.

### Example 1. Chemical Synthesis of N-benzamide Enaminone

The enaminone analogs were initially evaluated theoretically for drug-likeness (Table 1 and Table 2).

TABLE 1
Physicochemical Properties of Fluorinated N-Benzamide Enaminones

| | | | | | | | | P-Gp |
|---|---|---|---|---|---|---|---|---|
| Structure | Id | Mw^{a} | Clogp^{b} | #Hba^{c} | #Hbd^{d} | Tpsa^{e} | Logbb^{f} | Substrate^{g} |
| 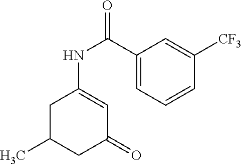 [SMILES: CC1CC(=O)C=C(NC(=O)c2cccc(C(F)(F)F)c2)C1] | IAB15 | 297.27 | 3.2371 | 5 | 1 | 46.17 | 0.372 | No |
| 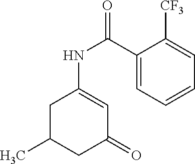 [SMILES: CC1CC(=O)C=C(NC(=O)c2ccccc2C(F)(F)F)C1] | IAA61 | 297.27 | 2.0271 | 5 | 1 | 46.17 | 0.32 | No |
| 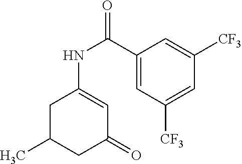 [SMILES: CC1CC(=O)C=C(NC(=O)c2cc(C(F)(F)F)cc(C(F)(F)F)c2)C1] | IAA65 | 365.27 | 4.2436 | 8 | 1 | 46.17 | 0.749 | No |
| 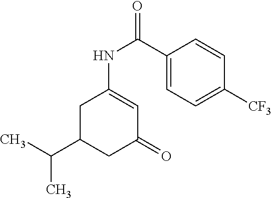 [SMILES: CC(C)C1CC(=O)C=C(NC(=O)c2ccc(C(F)(F)F)cc2)C1] | IAC41 | 325.33 | 4.1651 | 5 | 1 | 46.17 | 0.044 | No |
| 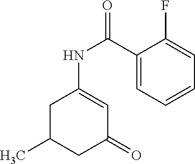 [SMILES: CC1CC(=O)C=C(NC(=O)c2ccccc2F)C1] | IAB67 | 247.26 | 1.9359 | 3 | 1 | 46.17 | 0.396 | No |
| 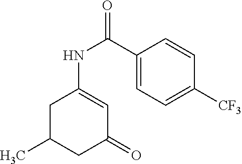 [SMILES: CC1CC(=O)C=C(NC(=O)c2ccc(C(F)(F)F)cc2)C1] | THA40 | 297.27 | 3.2371 | 5 | 1 | 46.17 | 0.073 | No |

TABLE 2
Physicochemical Properties of Meta-Fluorinated N-Benzamide Enaminones

| | | | | | | | | P-gp |
|---|---|---|---|---|---|---|---|---|
| Compounds | ID | MW^{a} | cLogP^{b} | HBA^{c} | #HBD^{d} | TPSA^{e} | LogBB^{f} | substrate^{g} |
| 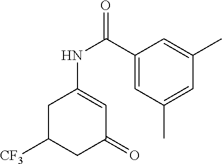 [SMILES: Cc1cc(C)cc(C(=O)NC2=CC(=O)CC(C(F)(F)F)C2)c1] | RHB62 | 311.30 | 2.975 | 3 | 1 | 46.17 | −0.094 | No |
| 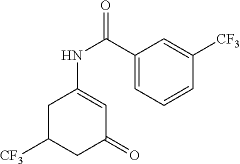 [SMILES: O=C1C=C(NC(=O)c2cccc(C(F)(F)F)c2)CC(C(F)(F)F)C1] | RHB95 | 351.25 | 3.202 | 3 | 1 | 46.17 | 0.292 | No |
| 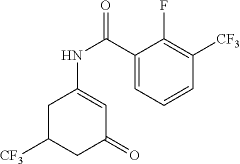 [SMILES: O=C1C=C(NC(=O)c2cccc(C(F)(F)F)c2F)CC(C(F)(F)F)C1] | RHB103 | 369.24 | 3.387 | 3 | 1 | 46.17 | 0.530 | No |
| 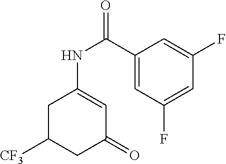 [SMILES: O=C1C=C(NC(=O)c2cc(F)cc(F)c2)CC(C(F)(F)F)C1] | RHB107 | 319.23 | 2.333 | 3 | 1 | 46.17 | 0.266 | No |
| 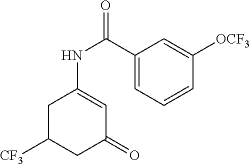 [SMILES: O=C1C=C(NC(=O)c2cccc(OC(F)(F)F)c2)CC(C(F)(F)F)C1] | RHB121 | 367.25 | 2.949 | 4 | 1 | 55.40 | −0.033 | No |
| 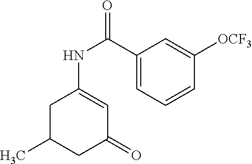 [SMILES: CC1CC(=O)C=C(NC(=O)c2cccc(OC(F)(F)F)c2)C1] | ABA58 | 313.28 | 3.312 | 4 | 1 | 55.40 | 0.172 | No |

Melting points (mp) were determined on a ThermoFisher digital capillary melting point apparatus and were uncorrected (Table 3). Reactions were monitored by 5×10 cm Whatman K6F silica gel thin layer chromatography (TLC) (60 Å; 250 μm layer thickness) fluorescent glass plates using a solvent system of ethyl acetate: hexane v/v (9:1). The ¹H, and ¹³C spectra were recorded on a Bruker Ultra Shield-400 MHz NMR spectrometer. The samples were dissolved in deuterated dimethylsulfoxide (DMSO-d₆). Gas chromatography mass spectroscopy analysis was performed on a Shimadzu QP-2010 PLUS Gas Chromatograph coupled with the Shimadzu QP-2010 SE Mass Spectrometer. Elemental analyses (C, H, N) were performed by Micro Analysis Inc. (Wilmington, DE, USA). The analytical results for the elements were within ±0.3% of the theoretical values. Starting materials 5-isopropylcyclohexane-1,3-dione and the substituted benzoyl chloride reagents were obtained from Sigma-Aldrich Chemical Company (Milwaukee, WI, USA) and used without further purification. 5-Methylcyclo-hexane-I, 3-dione was prepared by literature methods.

TABLE 3
Exemplary Meta-Fluorinated N-Benzamide Enaminones Synthesized

| Compound ID | Percent Yield (%) | Melting Point Range |
|---|---|---|
| RHB-59 | 62.5% | 160.4-161.5° C. |
| RHB-62 | 58.0% | 186.9-188.0° C. |
| RHB-77 | 43.5% | 151.0-151.5° C. |
| RHB-95 | 52.7% | 130.1-131.2° C. |
| RHB-99 | 46.3% | 165.4-166.2° C. |
| RHB-103 | 20.2% | 147.1-148.3° C. |
| RHB-107 | 54.0% | 170.2-170.8° C. |
| RHB-121 | 49.5% | 137.0-138.3° C. |
| RHB-119 | 61.6% | 148.3-149.2° C. |
| RHB-115 | 48.5% | 138.6-139.2° C. |
| RHB-127 | 42.3% | 170.3-171.2° C. |
| RHB-125 | 53.8% | 177.1-177.9° C. |

### Example 2. Synthesis of Enaminone Intermediate

General procedure A: 3-amino-5-trifluoromethylcyclohex-2-enone (2): To a 500 mL two-neck round bottom flask fitted with a condenser, Dean-Stark trap, and magnetic stirrer was added 300 mL of anhydrous benzene under nitrogen. The reaction flask was placed on an ice bath before adding 5-trifluoromethylcyclohexane-1, 3-dione (1, 13.40 g, 106.0 mmol) and ammonium acetate (16.34 g, 212.0 mmol). After stirring for 15 minutes, 5.5 mL of acetic acid was added dropwise, and the reaction mixture was brought to room temperature and allowed to stir for 30 minutes. The mixture was refluxed for 1 h and once cooled allowed to stir overnight at room temperature. The crude product precipitated and was collected via vacuum filtration and allowed to air dry. Once dried, the crude product was recrystallized from hot ethyl acetate as a yellow solid 80% (10.64 g), mp. 175-177° C. (lit. 173-174.5° C.). ¹H NMR (400 MHz, DMSO): δ, ppm 6.70 (2H, s), 4.90 (1H, s), 2.29-2.20 (1H, m), 2.08-1.96 (3H, m), 1.82-1.73 (1H, m), 0.97-0.94 (3H, m). ¹³C NMR (101 MHz, DMSO): δ, ppm 194.8, 166.9, 97.5, 44.8, 36.5, 29.3, 21.4. Anal. Calcd for C₇H₁₁NO: C, 67.2; H, 8.9; N, 11.2. Found: C, 67.2; H, 8.8; N, 11.2.

General procedure B: Amide coupling of amines to acyl chloride (3): To a 500 mL two-neck round bottom flask equipped with a condenser and magnetic stirrer was added 50 mL anhydrous tetrahydrofuran (THF) under nitrogen. After cooling on an ice bath, sodium hydride (960 mg, 39.94 mmol) was slowly added; followed by additional 40 mL of dry THF. 3-Amino-5-methylcyclohex-2-enone (2.500 g, 19.97 mmol) was added slowly over the course of 5 mins followed by 30 mL of dry THF. The reaction mixture was allowed to reflux for 20 mins. Once cooled to room temperature, the mixture was placed on an ice bath for 5 min before adding substituted benzoyl chloride (3.000 mL, 19.97 mmol) in 20 mL of dry THF via dropping funnel. The dropping funnel was rinsed with an additional 20 mL of dry THF. The reaction mixture was stirred in an ice bath for about 10 mins. Before removing the reaction mixture from the ice bath, a first aliquot was removed and analyzed. Reaction mixture was stirred at room temperature with monitoring done by TLC and GCMS until completion. Upon confirmation of completion, the reaction mixture was quenched with 100 mL deionized (DI) water and acidified with 10 mL of concentrated HCl. The aqueous solution was extracted with dichloromethane (2×85 mL) and the organic layer washed with 85 mL of 10% NaHCO₃ and 85 mL DI water. The organic phase was dried over MgSO₄, filtered, and concentrated in vacuo to yield a yellowish-white semi-solid residue that was triturated with anhydrous Et₂O. The white precipitate was collected via vacuum filtration. MP, CHN, GCMS and NMR was determined for each compound.

N-(5-methyl-3-oxocyclohex-1-en-1-yl)-3-(trifluoromethyl) benzamide-(3a, IAB15): Utilizing the general procedure outlined above, 3-amino-5-methylcyclohex-2-en-1-one (2.500 g, 19.97 mmol) was N-acylated using 3-trifluoromethyl benzoyl chloride (3.000 mL, 19.97 mmol) to give the titular compound as white powder in 42% yield (3.14 g), mp 181-182° C. ¹H NMR (400 MHz, DMSO): δ, ppm 10.18 (1H, s), 8.23-8.16 (2H, m), 7.98-7.95 (1H, m), 7.76 (1H, t, J=7.8 Hz), 6.78 (1H, d, J=1.3 Hz), 2.74 (1H, dd, J=4.1, 17.3 Hz), 2.23-2.13 (1H, m), 2.09-2.01 (1H, m), 1.04 (3H, d, J=6.4 Hz). ¹³C NMR (101 MHz, DMSO): δ, ppm 199.4, 165.8, 156.4, 135.4, 132.6, 130.2, 130.1, 129.8, 129.5, 129.2, 129.2, 129.1, 129.1, 125.7, 125.1, 125.1, 125.0, 125.0, 123.0, 112.0, 45.1, 40.0, 36.1, 29.2, 21.1, 0.5. Anal. Calcd for C₁₅H₁₄F₃NO₂: C, 60.6; H, 4.8; N, 4.7. Found: C, 60.6; H, 4.7; N, 4.8.

N-(5-methyl-3-oxocyclohex-1-en-1-yl)-2-(trifluoromethyl) benzamide-(3b, IAA61): Utilizing the general procedure outlined above, 3-amino-5-methylcyclohex-2-en-1-one (2.500 g, 19.97 mmol) was N-acylated using 2-trifluoromethyl benzoyl chloride (2.940 mL, 19.97 mmol) to give the titular compound as white powder in 21% yield (1.50 g), mp 180-181° C. ¹H NMR (400 MHz, DMSO): δ, ppm 10.44 (1H, s), 7.87-7.67 (4H, m), 6.68 (1H, s), 2.59 (1H, dd, J=3.8, 17.1 Hz), 2.32-2.02 (4H, m), 1.04-1.01 (3H, m). ¹³C NMR (101 MHz, DMSO): δ, ppm 199.3, 167.3, 155.9, 135.6, 135.6, 133.1, 131.0, 129.0, 126.9, 126.9, 126.8, 126.8, 126.8, 126.5, 126.1, 125.8, 125.5, 122.7, 111.7, 45.0, 40.0, 35.8, 29.1, 21.1, 0.5. Anal. Calcd for C₁₅H₁₄F₃NO₂: C, 60.6; H, 4.8; N, 4.7. Found: C, 60.6; H, 4.8; N, 4.7.

N-(5-methyl-3-oxocyclohex-1-en-1-yl)-3,5-bis(trifluoromethyl) benzamide-(3c, IAA65): Utilizing the general procedure outlined above, 3-amino-5-methylcyclohex-2-en-1-one (1.500 g, 11.98 mmol) was N-acylated using 3,5-bis-trifluoromethyl benzoyl chloride (2.200 mL, 11.98 mmol) to give the titular compound as white powder in 34% yield (1.51 g), mp 185-188° C. ¹H NMR (400 MHz, DMSO): δ, ppm 10.33 (1H, s), 8.52 (2H, s), 8.35 (1H, s), 6.77 (1H, d, J=1.3 Hz), 2.73 (1H, dd, J=4.1, 17.3 Hz), 2.41-2.14 (3H, m), 2.10-2.02 (1H, m), 1.05 (3H, d, J=6.4 Hz). ¹³C NMR (101 MHz, DMSO): δ, ppm 199.3, 164.3, 156.0, 136.7, 131.4, 131.1, 130.7, 130.4, 129.4, 129.3, 127.5, 126.1, 126.0, 125.9, 124.8, 122.1, 119.4, 112.4, 45.0, 40.0, 36.1, 29.1, 21.1, 0.4. Anal. Calcd for C₁₆H₁₃F₆NO₂: C, 52.6; H, 3.6; N, 3.8. Found: C, 52.6; H, 3.5; N, 3.8.

N-(5-isopropyl-3-oxocyclohex-1-en-1-yl)-4-(trifluoromethyl) benzamide-(3d, IAC41): To a 250 mL two-neck round bottom flask equipped with a condenser, pressure-equalizing dropping funnel, and magnetic stirrer was added 40 mL anhydrous tetrahydrofuran (THF) under nitrogen. After cooling on an ice bath, sodium hydride (562 mg, 23.40 mmol) was slowly added; followed by additional 30 mL of dry THF. 3-amino-5-isopropylcyclohex-2-enone (1.200 g, 7.800 mmol) was added slowly over the cause of 5 mins followed by 20 mL of dry THF. The reaction mixture was allowed to reflux for 20 mins. Once cooled to room temperature, the mixture was placed on an ice bath for 5 min before adding 4-trifluoromethylbenzoyl chloride (1.200 mL, 7.800 mmol) in 15 mL of dry THF via dropping funnel. The dropping funnel was rinsed with an additional 15 mL of dry THF. The reaction mixture was stirred in an ice bath for about 10 mins. Before removing the reaction mixture from the ice bath, a first aliquot was removed and analyzed. Reaction mixture was stirred at room temperature with monitoring done by TLC and GCMS until completion. Upon confirmation of completion, the reaction mixture was quenched with 80 mL deionized (DI) water and acidified with 8 mL of concentrated HCl. The aqueous solution was extracted with dichloromethane (2×75 mL) and the organic layer washed with 75 mL of 10% NaHCO₃ and 75 mL DI water. The organic phase was dried over MgSO₄, filtered, and concentrated in vacuo to yield a yellowish-white solid residue that was triturated with anhydrous Et₂O to afford the product in 52.9% (1.34 g) yield. 194.5-195.8° C. ¹H NMR (400 MHz, DMSO): δ, ppm 10.20 (1H, s), 8.11-8.07 (2H, m), 7.92-7.88 (2H, m), 6.82 (1H, d, J=1.4 Hz), 2.75 (1H, dd, J=3.8, 17.4 Hz), 2.42-1.79 (4H, m), 1.64-1.54 (1H, m), 0.94-0.89 (6H, m). ¹³C NMR (101 MHz, DMSO): δ, ppm 199.6, 166.2, 156.7, 138.3, 132.5, 132.2, 129.4, 125.9, 125.9, 125.8, 125.8, 125.6, 122.9, 112.0, 40.2, 31.8, 31.8, 19.9, 19.9, 0.5. Anal. Calcd for C₁₇H₁₈F₃NO₂: C, 62.8; H, 5.6; N, 4.3; F, 17.5 Found: C, 62.7; H, 5.6; N, 4.3; F, 17.5.

2-fluoro-N-(5-methyl-3-oxocyclohex-1-en-1-yl) benzamide-(3e, IAB67): Utilizing the general procedure outlined above, 3-amino-5-methylcyclohex-2-en-1-one (2.500 g, 19.97 mmol) was N-acylated using 2-fluoro benzoyl chloride (2.500 mL, 19.97 mmol) to give the titular compound as white powder in 25% yield (1.36 g), mp 126.7-127.7° C. ¹H NMR (400 MHz, DMSO): δ, ppm 10.25 (1H, s), 7.65-7.56 (2H, m), 7.37-7.29 (2H, m), 6.72 (1H, s), 2.65-2.58 (1H, m), 2.19-2.00 (2H, m), 1.04-1.00 (3H, m). ¹³C NMR (101 MHz, DMSO): δ, ppm 199.4, 164.5, 160.6, 158.1, 156.0, 133.7, 133.6, 130.5, 130.4, 125.1, 125.0, 124.7, 124.5, 116.8, 116.6, 111.6, 45.0, 40.0, 36.0, 29.1, 21.1, 0.5; Anal. Calcd for C₁₅H₁₄F₃NO₂: C, 68.0; H, 5.7; N, 5.7; F, 7.7 Found: C, 67.8; H, 5.9; N, 5.6; F, 7.8.

N-(5-methyl-3-oxocyclohex-1-en-1-yl)-4-(trifluoromethyl) benzamide-(3f, THA40): Utilizing the general procedure outlined above, 3-amino-5-methylcyclohex-2-en-1-one (2.500 g, 19.97 mmol) was N-acylated using 4-trifluoromethyl benzoyl chloride (2.960 mL, 19.97 mmol) to give the titular compound as white powder in 48% yield (2.85 g), mp 202-203° C. ¹H NMR (400 MHz, DMSO): δ, ppm 10.21 (1H, s), 8.10-8.06 (2H, m), 7.93-7.89 (2H, m), 6.78 (1H, s), 2.74 (1H, dd, J=4.1, 17.4 Hz), 2.40-2.27 (2H, m), 2.21-2.02 (2H, m), 1.05 (3H, d, J=6.4 Hz). ¹³C NMR (101 MHz, DMSO): δ, ppm 199.4, 166.2, 156.3, 138.3, 138.3, 132.5, 132.1, 129.4, 125.9, 125.9, 125.8, 125.8, 125.6, 122.9, 112.0, 45.1, 40.0, 36.0, 29.1, 21.1, 0.5. Anal. Calcd for C₁₅H₁₄F₃NO₂: C, 60.6; H, 4.8; N, 4.7. Found: C, 60.4; H, 4.5; N, 4.7.

### Example 3. Cell Culture, Differentiation and Infection of ND7/23 Cells

ND7/23 cells are derived from the fusion of mouse neuroblastoma and rat dorsal root ganglion cells, which generates a more homogeneous sensory neuron-like cell population. ND7/23 cells were purchased from Sigma-Aldrich (RRID:CVCL_4259). Culture and differentiation of ND7/23 cells was performed as previously described. Briefly, ND7/23 cells were cultured in DMEM/F12 culture media (Millipore, Cat. #DF-041-B), supplemented with 0.5% fetal bovine serum (Invitrogen, Cat. #10437010), db-cAMP (1 mM, Sigma-Aldrich, Cat. #D0627), and nerve growth factor (NGF, 50 ng/mL, Sigma-Aldrich, Ca. #N2513). To remove any proliferating cells, cultures were treated with uridine (20 μM, Sigma-Aldrich, Cat. #U3003) and fluorodeoxyuridine (20 μM, Sigma-Aldrich, Cat. #F0503). Cells were maintained in differentiation media without uridine and fluorodeoxyuridin after induction of differentiation. ND7/23 cells were maintained in differentiation media for ≤6 days. HEK 293 cells that were stabled transfected with the human Cav3.2 and Cav3.3 channel subunits were a gift of Dr. E. Perez-Reyes (University of Virginia). Cav3.2 (or Cav3.3)-stabled transfected HEK 293 cells were culture in DMEM/F12 culture media, supplemented with 5% fetal bovine serum, 50 U/mL penicillin and 50 μg/mL streptomycin. ND7/23 and HEK 293 cells were grown in an incubator at 37° C. in the presence of 5% CO₂/95% air humidified atmosphere. Cells passaged less than 20 times were used in this work. Cells were grown in poly-D-lysine-coated glass coverslips for electrophysiological recordings.

### Example 4. Electrophysiology

Whole cell recordings were performed as previously described. A Nikon Eclipse Ti inverted microscope equipped with Hoffman optics and epifluorescence filters was used to visualize individual cells. Recordings were performed at room temperature (22-24° C.) using glass electrodes made from thin wall borosilicate glass (3-5 MΩ). For electrophysiological recordings of inward Ca²⁺ currents, outward K⁺ currents were eliminated by equimolar substitution of K⁺ with Cs⁺ in the pipette solution, whereas inward Na⁺ currents were eliminated by the use of TEA ions in the external solution, which are unable to go through voltage-gated Na⁺ channels. The pipette solution consisted of (in mM) CsCl (120), MgCl₂(2), HEPES (10), EGTA (10), ATP (1), and GTP (0.1), pH 7.4 with CsOH. The composition of the normal external saline used for measurements of Ca²⁺ currents was (in mM): tetraethylammonium chloride (TEACl, 145), CaCl₂ (10), MgCl₂(1), HEPES (10), and glucose (5), pH 7.4 adjusted with CsOH. Ca²⁺ currents were generated by applying a 200-500 ms-depolarizing step to various potentials from a holding potential of −100 mV. The external solution used to measure Na⁺ currents contained (in mM) NaCl (145), KCl (5.3), CaCl₂ (0.5), MgCl₂ (5.7), HEPES (13), and glucose (5), pH 7.4 adjusted with KOH. The composition of the CsCl-pipette solution used to record Na⁺ currents was the same describe above. Na⁺ currents were generated by applying a 20 ms-depolarizing step to various potentials from a holding potential of −100 mV. To assess drug-evoked changes on specific ionic conductances, current amplitudes were expressed as current densities as previously reported. To assess current densities, cell size was normalized by dividing current amplitudes by cell capacitance. Cell capacitance was determined by integrating the transient current evoked by a 10-mV voltage step from a holding potential of −60 mV.

A MULTICLAMP 700A amplifier and PCLAMP software (Axon Instruments) was used to deliver voltage commands and to perform data acquisition and analysis. Pipette offset, whole cell capacitance, and series resistance were compensated automatically with the MultiClamp 700B Commander. Only cells with stable seals and series resistance (≤10 MΩ) were analyzed. Sampling rates were between 5 and 10 kHz. Leak currents were subtracted online using a P/4 protocol. The current-voltage relationship was obtained by applying 200 ms-depolarizing steps to various potentials from a holding potential of −100 mV and plotting the value of the voltage step versus the amplitude of the transient current. The activation and inactivation time constants (τ) were obtained by fitting the rising or decay portion of the transient currents with one exponential function in the form I(t)=A exp(−t/r), where A is peak current and r is the time constant. Steady-state inactivation was assessed by applying a 1000 ms pre-pulse between −100 and −30 mV, followed by a test pulse to −20 mV for 200-500 ms. Steady-state inactivation curves were fitted with a Boltzmann equation using normalized current values I/Iₘₐₓ=(1+exp(V−V_{1/2})/kᵢ)⁻¹, where I is the current at membrane voltage V, Imax is maximal current with the −100 mV conditioning pre-pulse, V_{1/2} is the half-activation voltage, and k is the slope factor. The time dependence of recovery from short-term inactivation was determined by applying a 200 ms pre-pulse to +30 mV from a holding potential of −100 mV, followed by an identical test potential, but with increased duration of the inter-pulse interval (0-1500 ms).

For electrophysiological recordings, compounds were dissolved in DMSO to prepare 50 mM concentrated stock solutions. The range of final concentrations tested were between 1 and 100 μM. Thus, the maximal volume of DMSO never exceeded 2 μL/1 mL bath solution, which had no effect on the recorded currents. Because of difficulties in washing off the drugs once applied, the Ca²⁺ currents were recorded in 5-6 cells. This was followed by a 5 min exchange of the bath solution containing specific compounds and subsequent recording of Ca²⁺ currents in the presence of the drugs. This experimental procedure was performed in at least 2 coverslips for each concentration tested.

### Example 5. Data Analysis

Values are presented as mean±SEM where indicated. Statistical analyses of normally-distributed samples consisted of t-test for pairwise comparisons or one-way ANOVA followed by post hoc analysis using Tukey's honest significant difference test for unequal n for comparisons between multiple groups using SigmaStat software. Throughout, p≤0.05 was regarded as significant.

### Example 6. Chemistry Results

For the design of the novel N-benzamide enaminone analogs, the recommended cutoffs of the SimulationPlus® physicochemical properties prediction tools MedChem Designer and ADMET Predictor 10.0 were used. All the compounds passed the test for drug-likeness including the measure of crossing the blood brain barrier (BBB) and likelihood of being a substrate of the P-glycoprotein (P-gp) efflux protein. These characteristics highlight the importance of each property as it relates to the chemistry of the compounds.

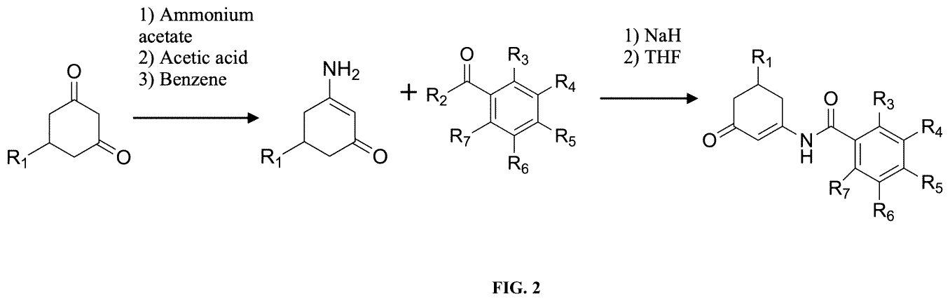

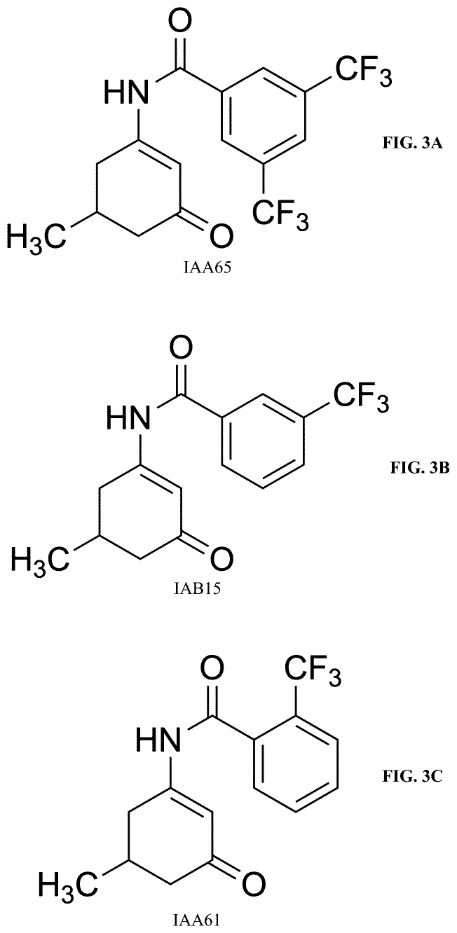

Once the compounds were confirmed to be druggable, next, the synthesis of the analogs as previously described was completed. The target N-benzamide enaminones were synthesized via a base catalyzed N-acylation reaction, which generally begins with amination of the respective β-diketones (FIG. 2)

The trifluoromethylation reaction was carried out by refluxing the diketone with ammonium acetate as both the amine source and base to generate the enaminone intermediate. The subsequent reaction was to generate the enaminone anion using two equivalence of sodium hydride in anhydrous tetrahydrofuran (THF). Due to the poor nucleophilicity of the enaminone system which is also known as a vinylogous amide, a super base such as sodium hydride was needed for N-deprotonation. This was achieved by refluxing the enaminone intermediate for about 20 minutes in sodium hydride with anhydrous THF. N-acylation with respective acyl chloride gave the desired enaminone N-benzamide analogs. Thin Layer Chromatography (TLC) and Gas Chromatography Mass Spectroscopy (GCMS) methods were used to monitor the reaction progress. Completion of the reactions (from the TLC and GCMS results) led to quenching of the reaction with water and concentrated HCl. The desired product was obtained from a liquid-liquid extraction with dichloromethane three times and washed with DI water. The organic mixture was dried over anhydrous magnesium sulfate and concentrated in vacuo to yield the N-benzamide analogs. The N-benzamide compounds were purified by using a moderate polar solvent system on the CombiFlash Redisep chromatography instrument. Cold trituration was done by washing the partially pure compound with anhydrous ether to remove any residual impurities.

The physicochemical properties of the enaminones analogs tested in this work are presented in Table 1. The molecular weight of the synthetized compounds was <400 g/mol, ideal for potential drug molecules targeting the central nervous system. The values of cLogP (denote a high octanol-water partition coefficient), measure of the lipophilicity of a drug molecule. The number of O and N hydrogen bond acceptors (HBA) and number of OH and NH hydrogen bond donors (HBD) was also acceptable with values of <10 and <5 for drugs targeting the CNS, respectively. The topological surface area (TPSA), a qualitative estimate of crossing the BBB was less than 70 Å, as required for drugs acting on the CNS. LogBB represents the predicted logarithm of the brain/blood partition coefficient, which can affect permeation through the BBB. BBB permeable drugs should have LogBB values of ≥−0.3. The compounds tested have a low effect on F (Pgp_Inh), which could decrease drug efficiency by promoting drug extrusion from neuronal cells.

### Example 7. Biological Evaluation

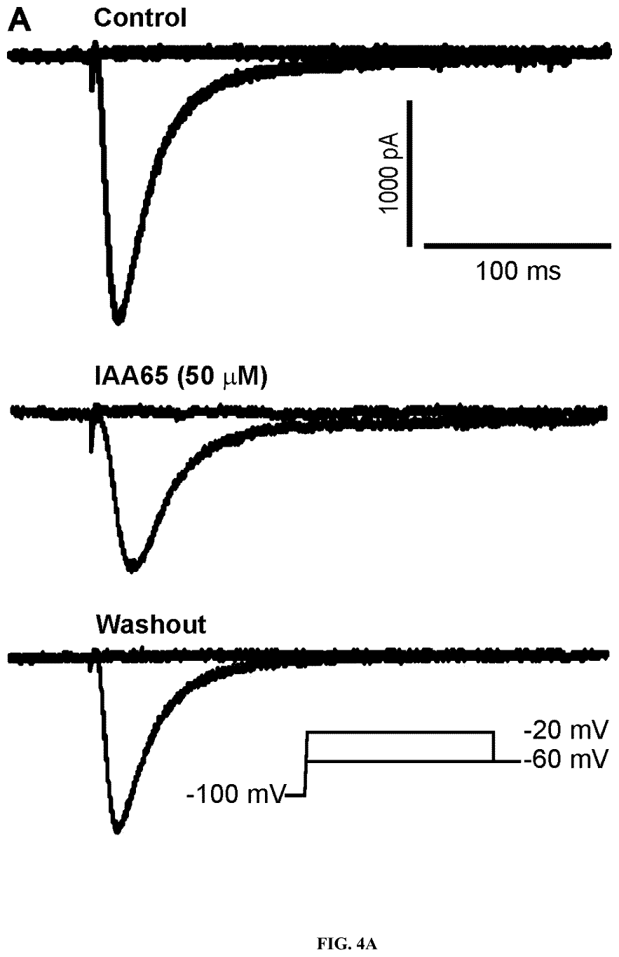

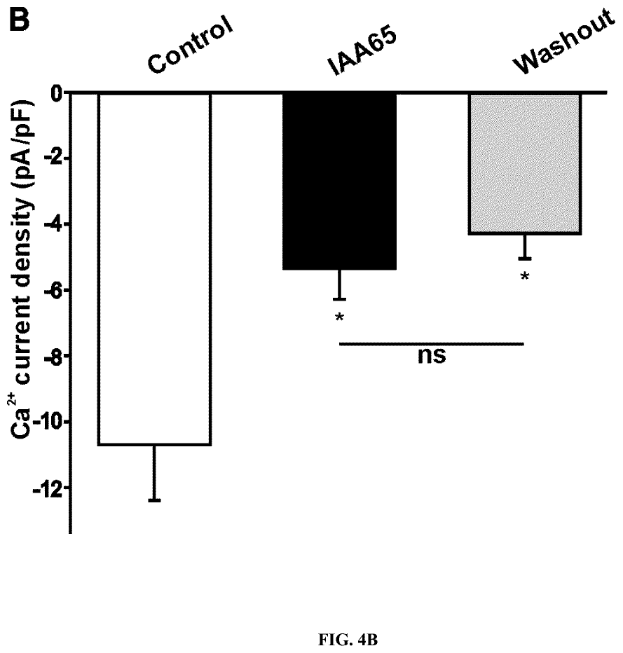

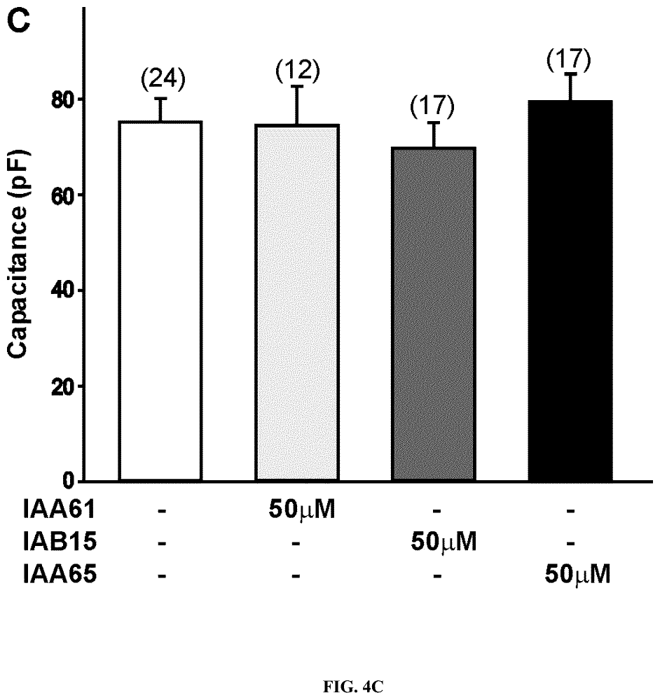

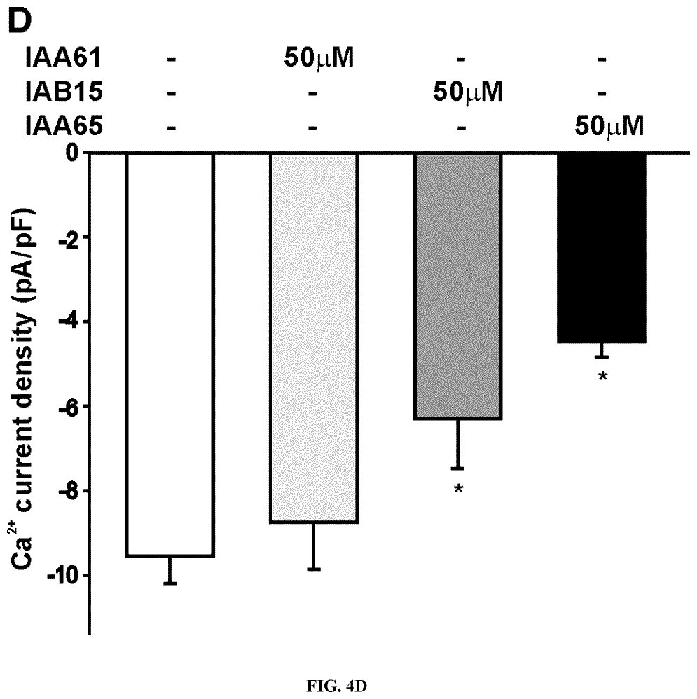

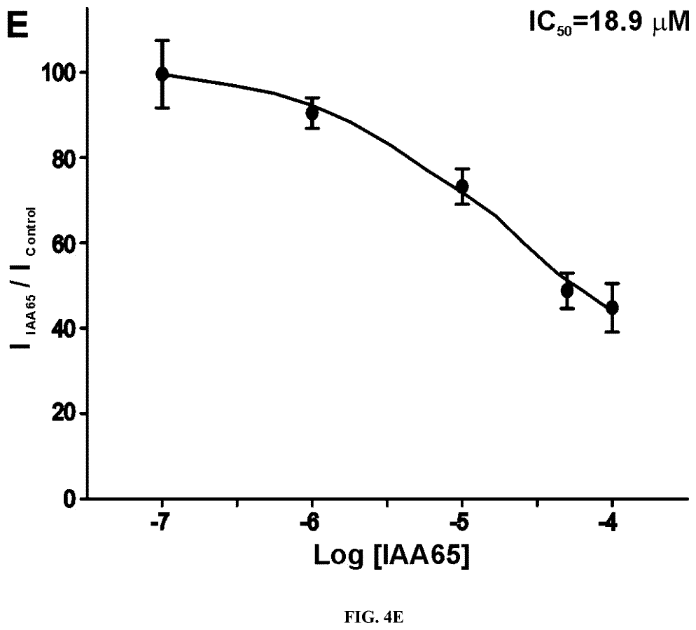

As previously reported, differentiated ND7/23 cells are sensory-like neurons that express a variety of voltage-activated ion channels. Differentiated ND7/23 cells express a robust T-type Ca²⁺ current (FIG. 4A). In −40% of recorded cells it was also observed the presence of high-voltage activated (HVA) Ca²⁺ currents (not shown). Initially, the ability of trifluoromethylated enaminone analogs was assessed to washout following inhibition of T-type Ca²⁺ currents. Typical T-type Ca²⁺ currents generated in ND7/23 cells before and after treatment with 50 μM IAA65 are presented in FIG. 4A. Acute treatment of ND7/23 cells with 50 μM IAA65 evoked a significant reduction T-type Ca²⁺ current densities (FIG. 4B). However, after 5 min washout, the effect of IAA65 was still evident with no significant recovery of T-type Ca²⁺ currents (FIG. 4A-B), this suggest that the effect of the trifluoromethylated enaminone analog was not reversed by the drug removal. This phenomenon is most likely due to the compounds high lipophobic characteristics. Since the effect of IAA65 on T-type Ca²⁺ currents were not reversed upon drug washout, all further experiments were conducted by independent recordings in control cells and cells treated with the trifluoromethylated enaminone analogs. Next, the effect of three trifluoromethylated enaminone analogs (IAA61, IAB15, and IAA65) were compared on differentiated N7/23 cells (Table 1). Exposure of differentiated ND7/23 cells to 50 μM of IAA61, IAB15, or IAA65 had no effect on cell capacitance (FIG. 4C). Both IAB15 and IAA65 are enaminone analogs with a 5-methyl group at the cyclic enaminone and a mono-meta trifluoromethyl and a 3,5-bis methyl groups at the meta position of the aromatic ring, respectively (Table 1). However, treatment of differentiated ND7/23 cells with 50 μM IAB15 or IAA65 caused a significant reduction in the current density generated by T-type Ca²⁺ channel activation (FIG. 4D). There was no effect of 50 μM IAA61 on the current density generated by T-type Ca²⁺ channel activation. IAA61 contains a trifluoromethyl group in the ortho position of the aromatic ring and a methyl group in the cyclic enaminone (Table 1). The effect of IAA65 on T-type Ca²⁺ channels was concentration-dependent as represented in FIG. 4E with an IC50=10.6 μM. Thus, the results suggest that enaminone analogs with a trifluoromethyl group in the meta position are more effective in blocking T-type Ca²⁺ channels, than those with similar group in the ortho position.

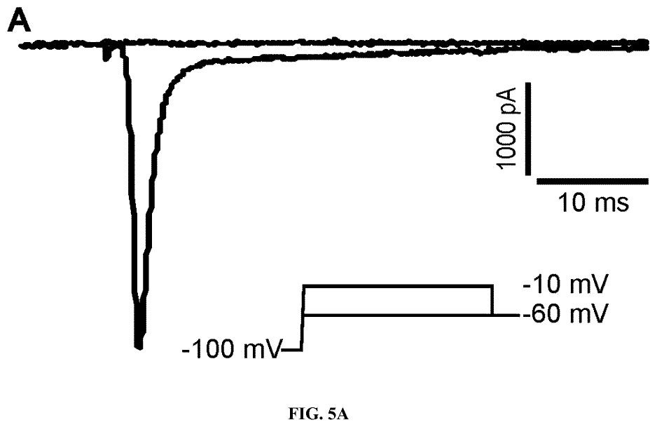

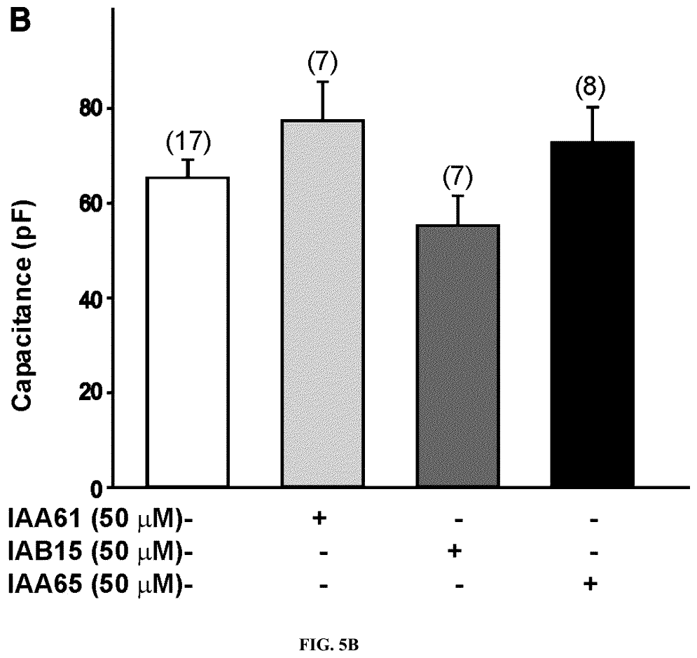

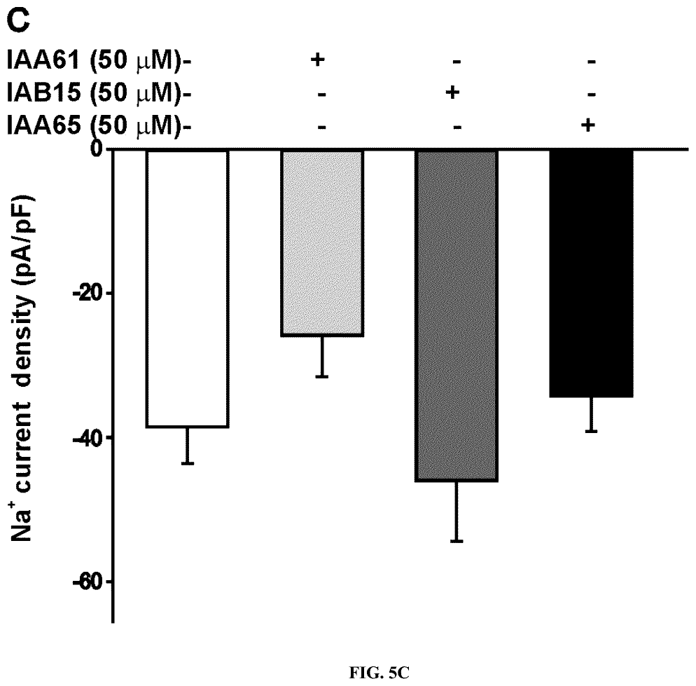

The possible effect of the trifluoromethylated enaminone analogs IAA61, IAB15, and IAA65 on voltage-activated Na⁺ channels (FIG. 5) was tested. A typical example of Na⁺ currents generated in differentiated ND7/23 cells by a depolarizing voltage step to −10 mV from a holding potential of −100 mV is represented in FIG. 5A. Treatment of differentiated ND7/23 cells with 50 μM of IAA61, IAB15, or IAA65 had no noticeable effect on cell capacitance or Na⁺ current density (FIG. 5B-C).

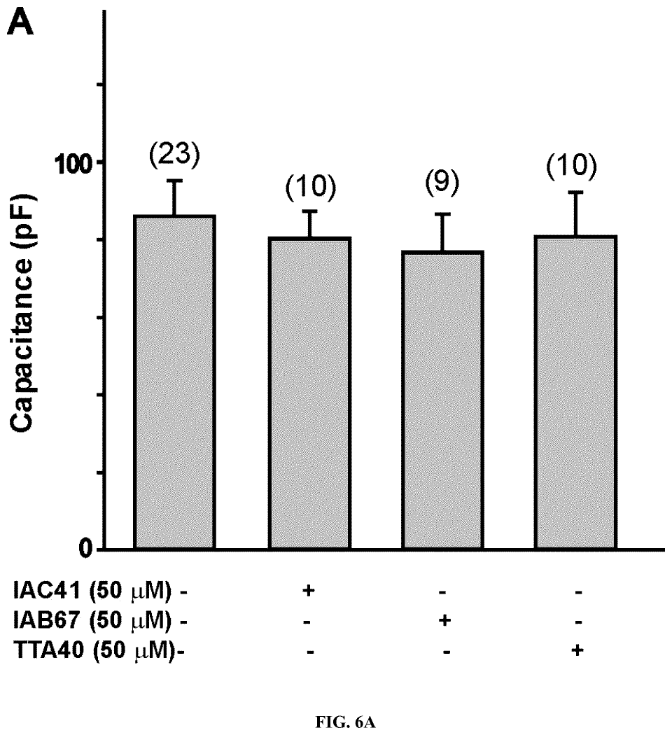

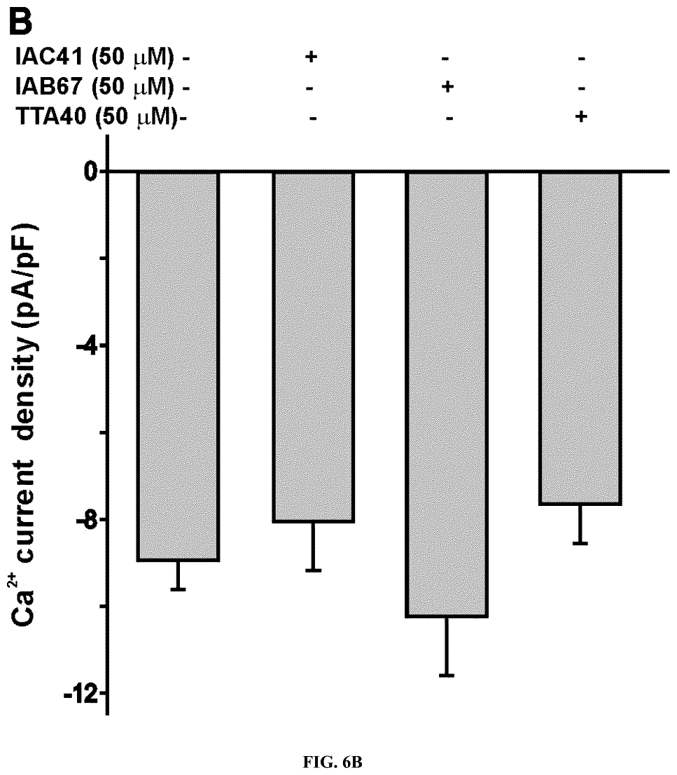

The effect of other enaminone analogs, including IAC41 (with a trifluoromethyl group in the para position and a 5′-isopropyl group in the cyclic enaminone), IAB67 (with a fluorine group in the ortho position and a 5′-methyl group in the cyclic enaminone), and THA40 (with a trifluoromethyl group in the para position and a 5′-methyl group in the cyclic enaminone) was tested on T-type Ca²⁺ channels (Table 1). None of the tested compounds has any effect on cell capacitance or the current density generated by T-type Ca²⁺ channel activation (FIG. 6A-B).

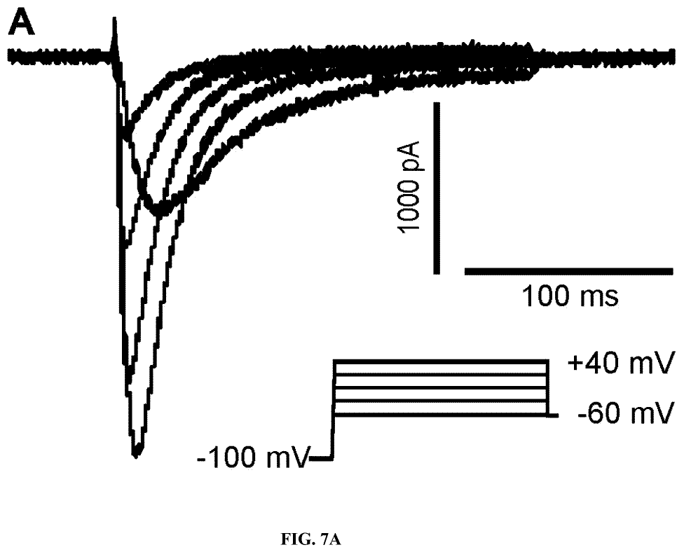

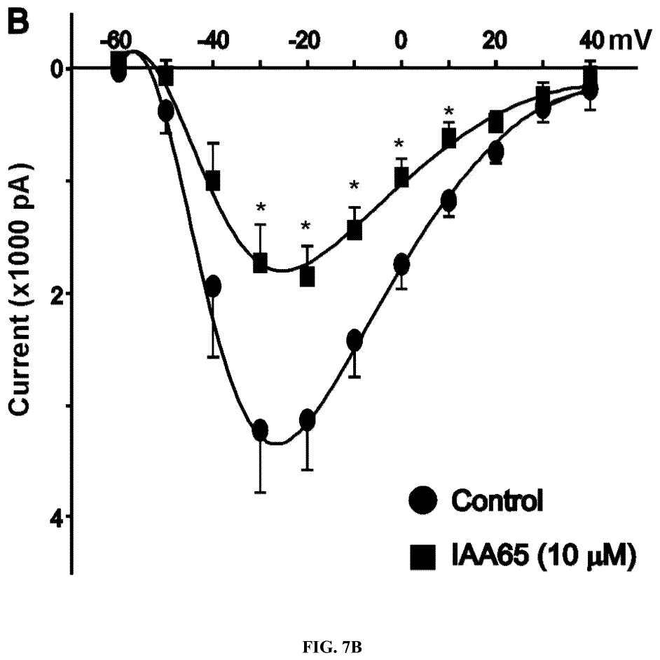

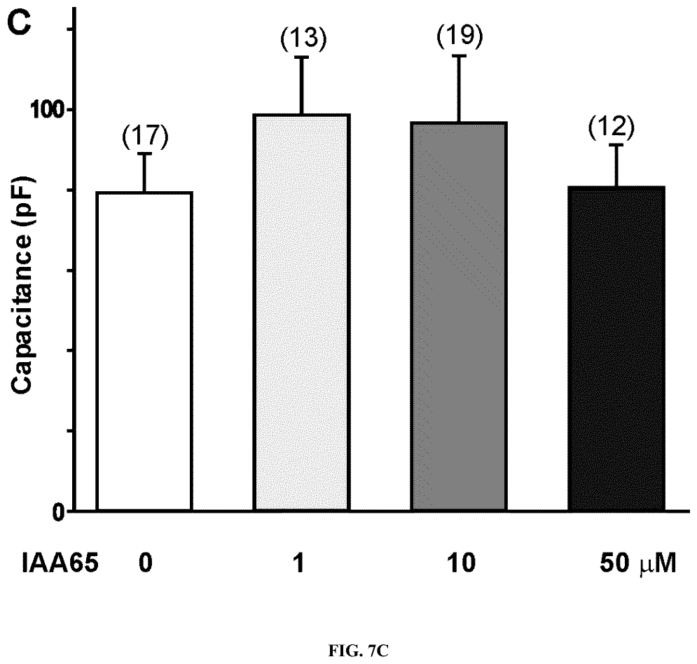

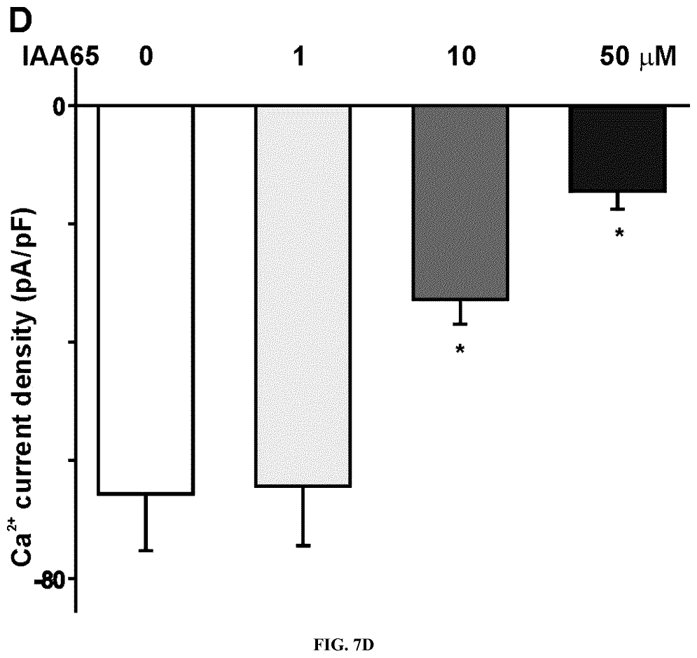

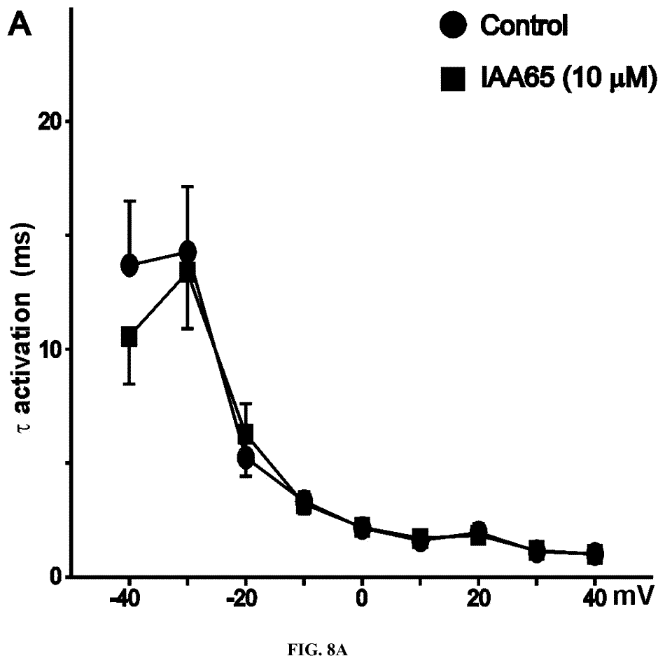

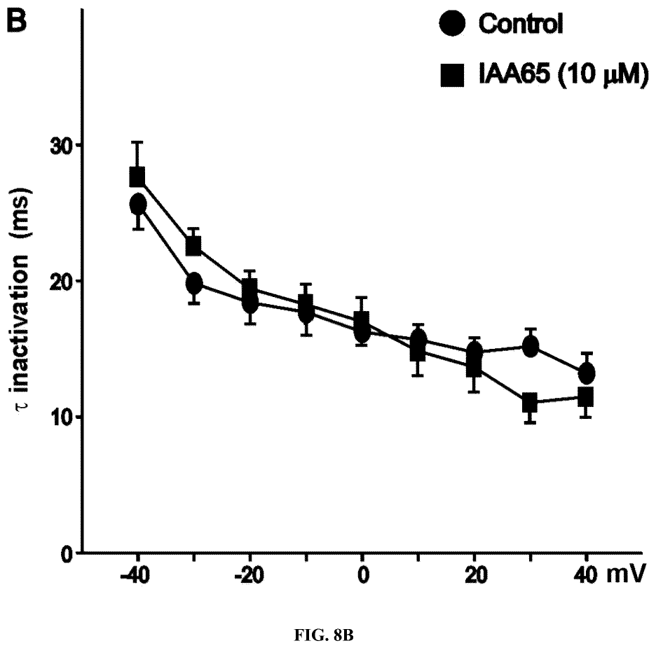

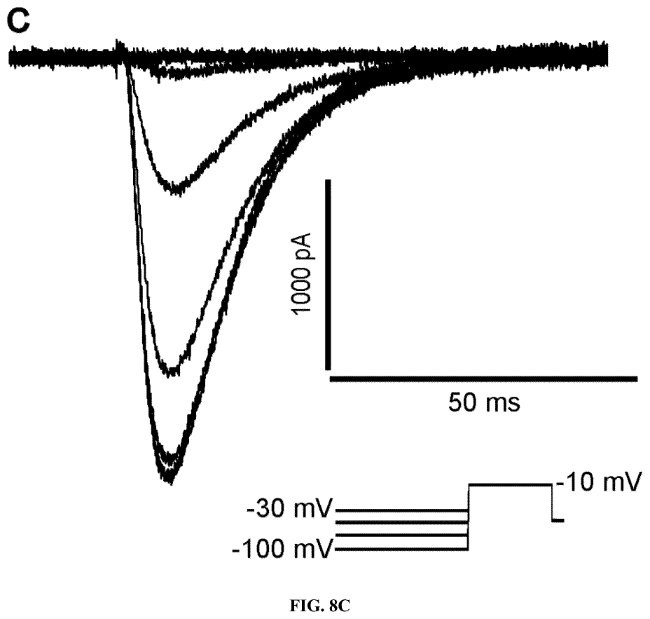

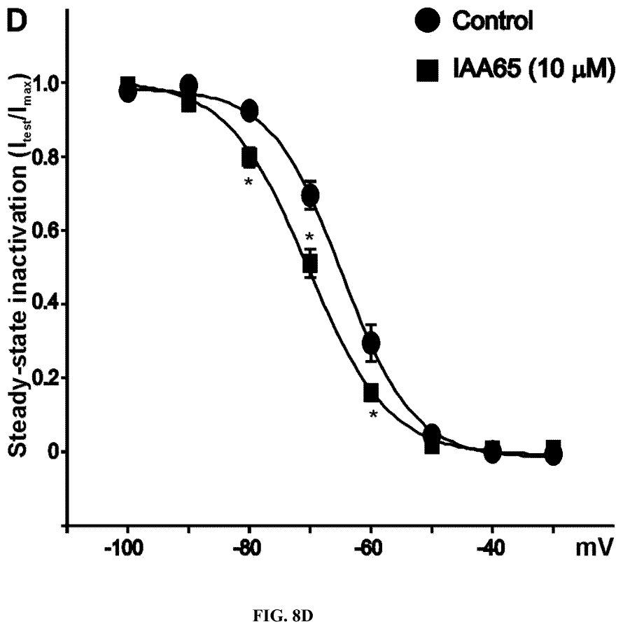

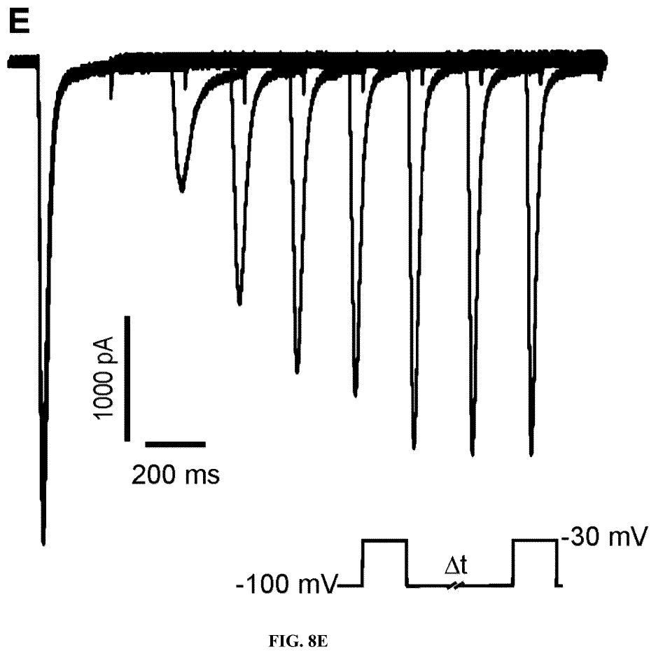

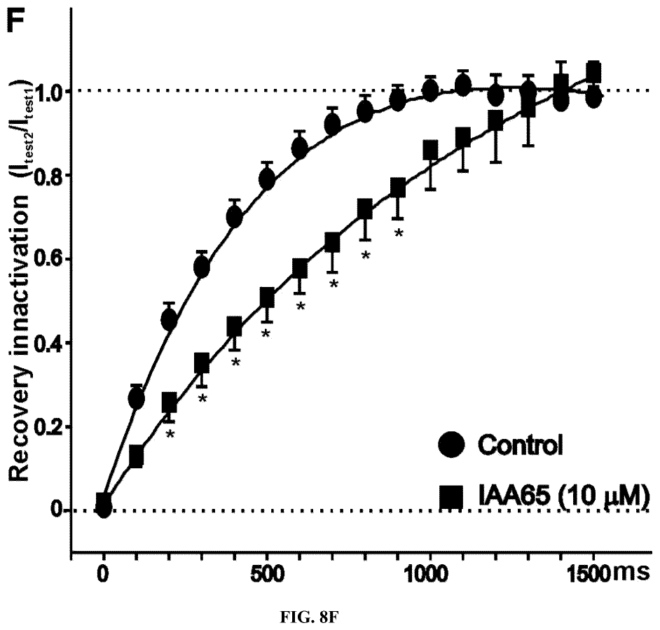

In order to investigate the mechanism of action of meta-trifluoromethyl substituted analogs on T-type Ca²⁺ channels, IAA65 was tested on Cav3.2 stable-transfected HEK 293 cells. As shown in FIG. 7A, Cav3.2 stable transfected HEK 293 cells generate a large transient inward current. The peak current generated by activation of T-type Ca²⁺ channels reaches a peak between −30 to −20 mV. Treatment of Cav3.2 stable transfected HEK 293 cells with 10 μM IAA65 evokes a significant reduction in the peak current without significant changes in the voltage-dependence (FIG. 7A-B). Although increasing concentrations of IAA65 (1-50 μM) had no effect on cell capacitance (FIG. 7C), it resulted in a significant reduction in the current density generated by activation of T-type Ca²⁺ channels (FIG. 7D). The activation and inactivation time constant (τ) of the transient current generated in Cav3.2 stable transfected HEK 293 cells was tested. The activation τ was voltage dependent (FIG. 8A) and presented a significant reduction between −40 and +40 mV. However, there were no significant changes in the activation τ between control and IAA65 (10 μM) treated cells. Similarly, the inactivation τ was voltage-dependent and underwent a significant reduction between −40 and +40 mV (FIG. 8B). Treatment of Cav3.2 stable transfected HEK 293 cells with 10 μM IAA65 had no effect on the inactivation τ (FIG. 8B). The effect of IAA65 on steady-state inactivation was tested. Steady-state inactivation was assessed by holding the membrane potential to various levels before applying a test potential to activate T-type Ca²⁺ channels (FIG. 8C). Plot of the normalized current as a function of the pre-test membrane potential generates a sigmoidal curve that was fitted with the Boltzmann equation (FIG. 8D). The fitted values of V_{1/2} in control and IAA65-treated cells were −64.8±1.3 (n=12) and −70.2±1.0 mV (n=16), respectively (p≤0.05), whereas kᵢ values were 5.2±0.2 (n=12) and 6.0±0.3 (n=16), respectively (p≤0.05). As represented in FIG. 8D, treatment of Cav3.2 stable transfected HEK 293 cells with 10 μM IAA65 caused a leftward shift in the sigmodal curve, indicating an increased number of inactivated channels (and therefore unable to open). Thus, it appears that IAA65 evokes a significant increase in the inactivation state of T-type Ca²⁺ channels. This resulted in a significant increase in the recovery time from short term inactivation in cells treated with IAA65 (10 μM) as represented in FIG. 8E-F. The recovery time from short term inactivation was assessed by applying 200 ms pre-pulse to +30 mV from a holding potential of −100 mV, following an identical test potential, but with increasing duration of the inter-pulse interval (Δt, 0-1500 ms, see Methods). As represented in FIG. 8F, treatment of Cav3.2 stable transfected HEK 293 cells with 10 μM IAA65 caused a rightward shift in the recovery time from inactivation (control cells recovery time=514.5±37.8 ms vs. IAA65-treated cells recovery time=963.0±30.1 ms). These findings suggest that IAA65 prolong the inactivation of T-type Ca²⁺ currents generated by the Cav3.2 subunits.

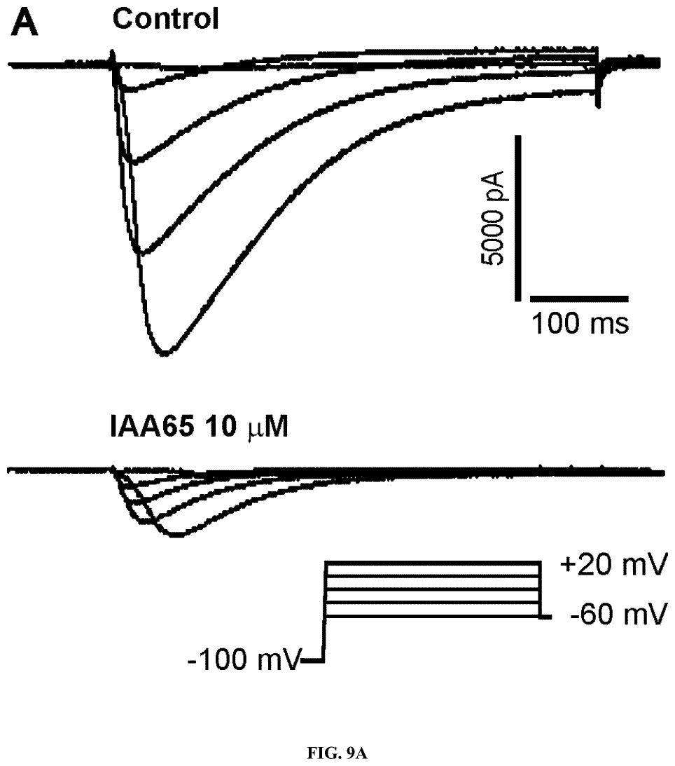

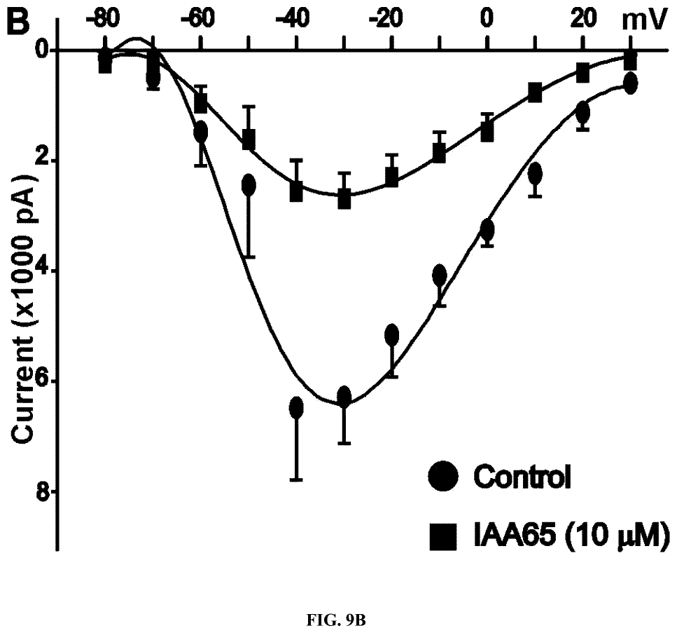

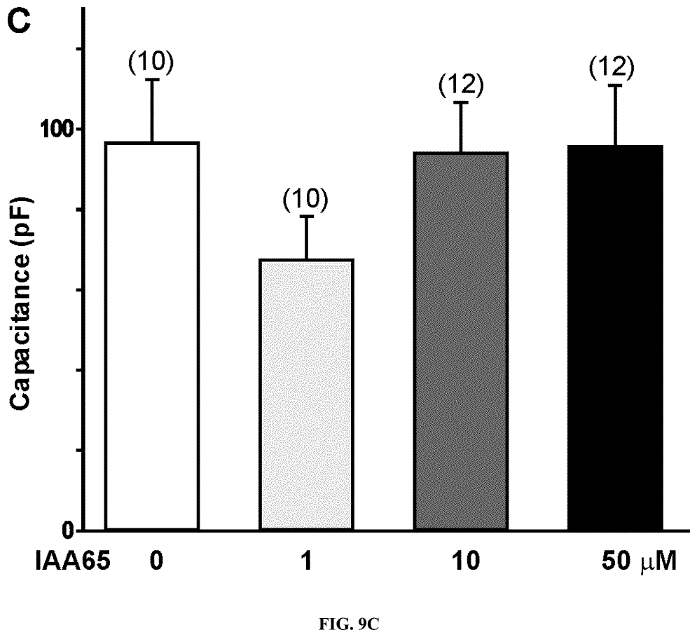

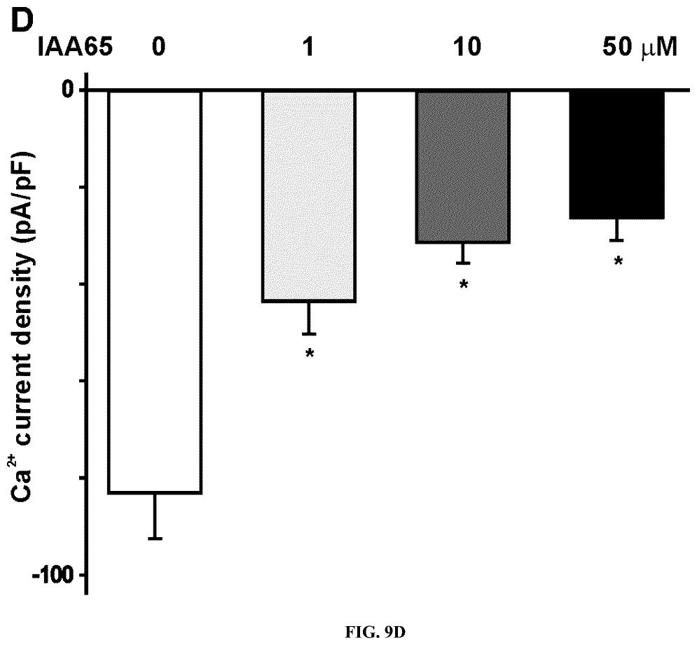

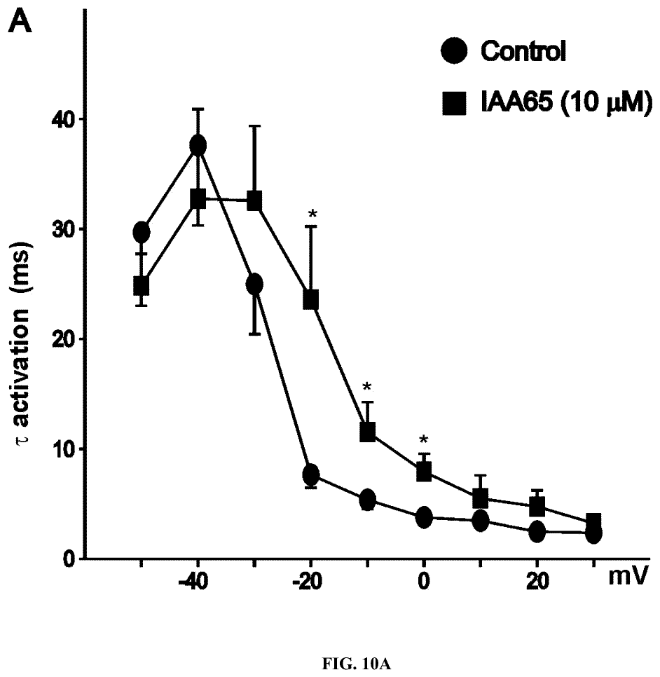

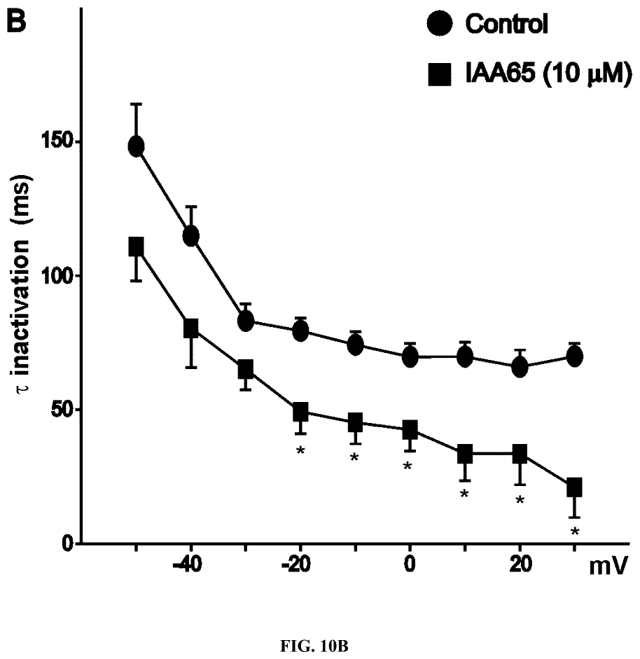

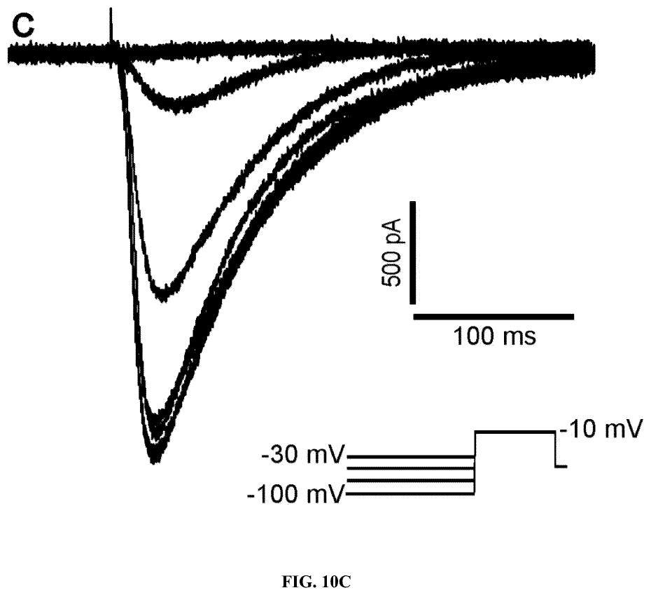

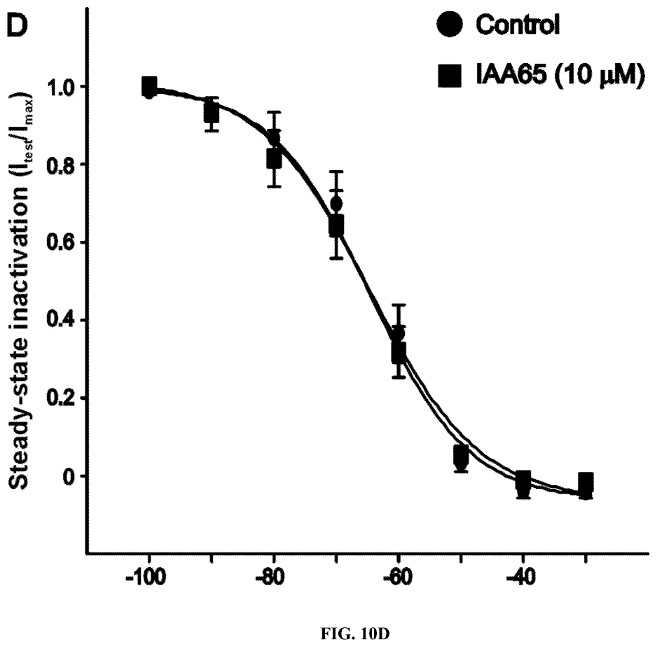

Therefore, the question arises on whether IAA65 have similar mechanism of action on T-type Ca²⁺ currents generated by the Cav3.3 subunits. As previously reported, T-type Ca²⁺ currents generated by Cav3.3 subunits present a slower activation and inactivation time constants. As represented in FIG. 9A, T-type Ca²⁺ currents generated by Cav3.3 subunits result in a transient conductance that undergo activation and inactivation ˜500 ms compared with currents generated by the activation of Cav3.2 subunits that last <200 ms (compare FIGS. 7A and 9A). T-type Ca²⁺ currents generated by Cav3.3 subunits peak at voltages between ˜40 and ˜30 mV (FIG. 9B). Treatment of Cav3.3 stable transfected HEK 293 cells with 10 μM IAA65 evokes a considerable reduction in the voltage-current relationship (FIG. 9A-B). Testing of different concentrations on IAA65 (1-50 μM) reveals no effect on call capacitance but it causes a significant reduction in the current density generated by the activation of T-type Ca²⁺ channels (FIG. 9C-D). The effect of 10 μM IAA65 on the activation and inactivation τ (FIG. 10A-B) was tested. Treatment of Cav3.3 stable transfected HEK 293 cells with 10 μM IAA65 caused a rightward shift in the activation τ (FIG. 10A), indicating that this fluorinated enaminone delays the opening of Cav3.3 T-type Ca²⁺ channels. On the other hand, treatment of Cav3.3 stable transfected HEK 293 cells with 10 μM IAA65 caused a reduction in the inactivation τ (FIG. 10B), indicating that this fluorinated enaminone increases the inactivation (closure) of Cav3.3 T-type Ca²⁺ channels. There was no effect of 10 μM IAA65 on the steady-state inactivation of T-type Ca²⁺ currents generated by the Cav3.3 subunits compared to control cells (FIG. 10D). There were no significant differences in the fitted values of V_{1/2} and kᵢ in control and IAA65-treated cells [Control V_{1/2}2=−65.6±3.1 (n=9), IAA65 V_{1/2}=−58.5±2.1 mV (n=9), p>0.05; Control kᵢ=5.6±0.3 (n=9), IAA65 kᵢ=5.3±0.6 (n=9), p>0.05].

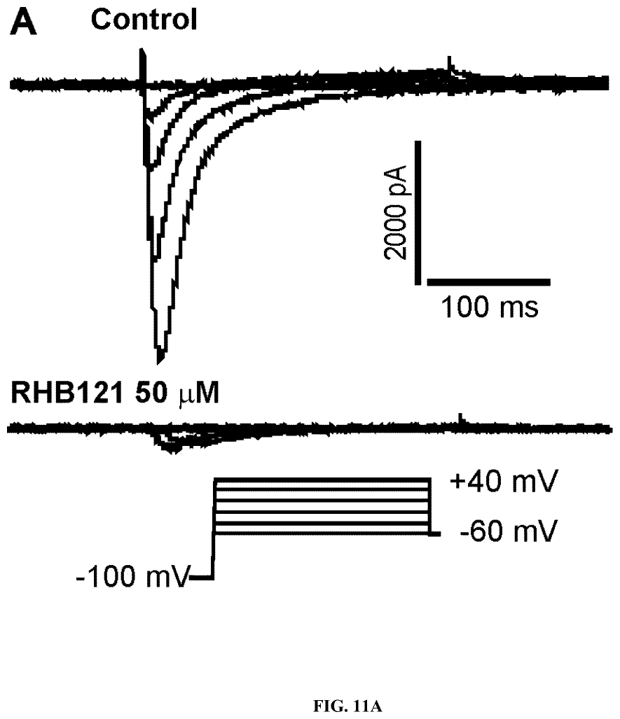

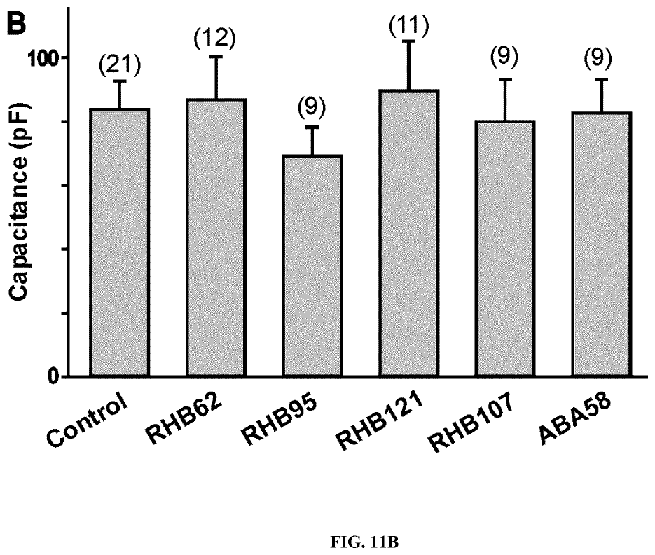

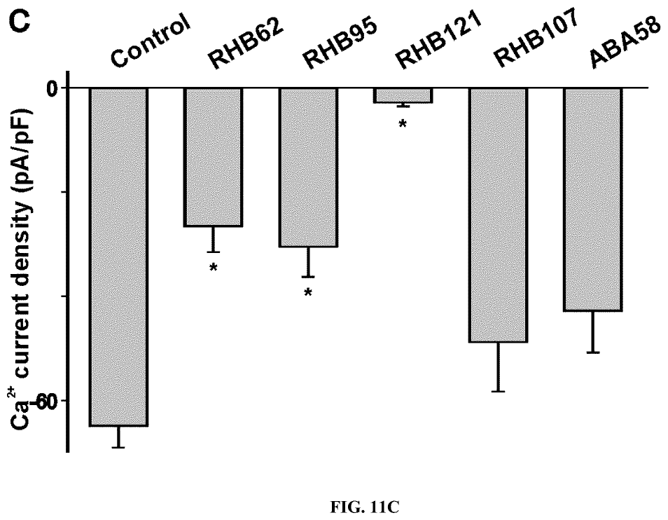

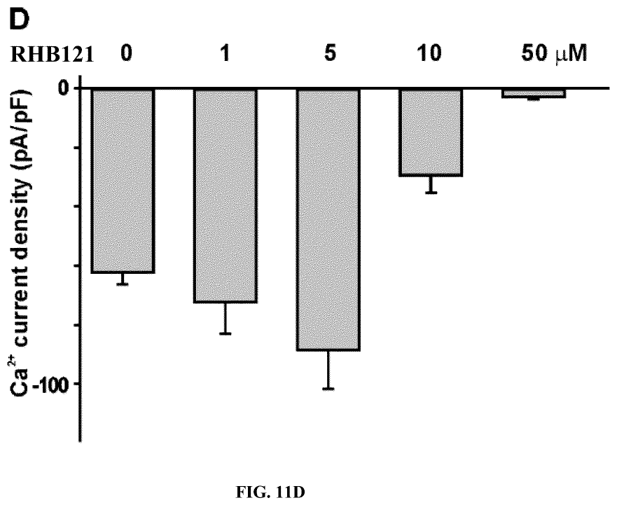

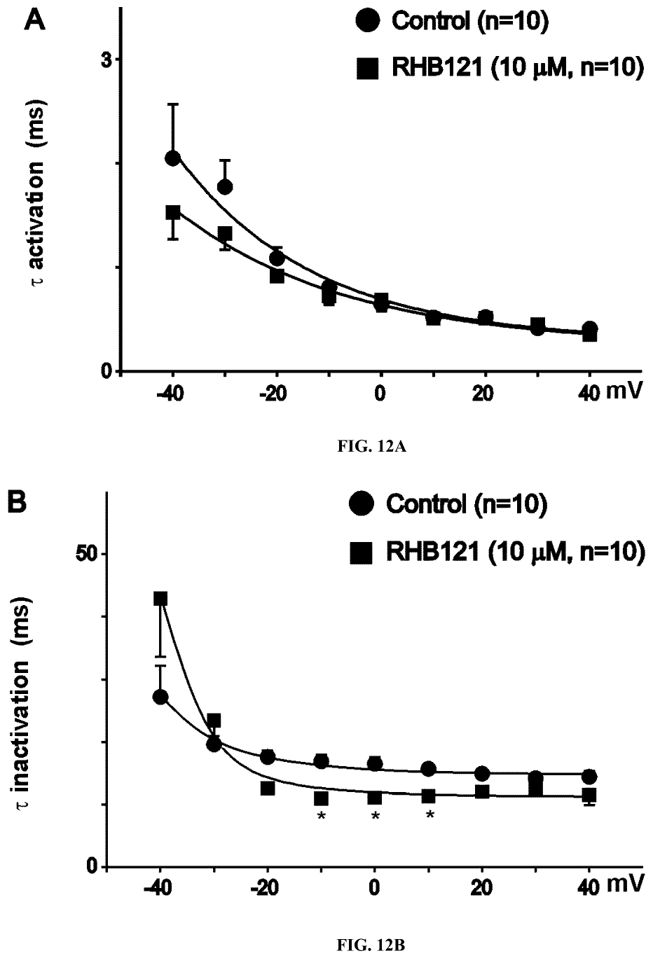

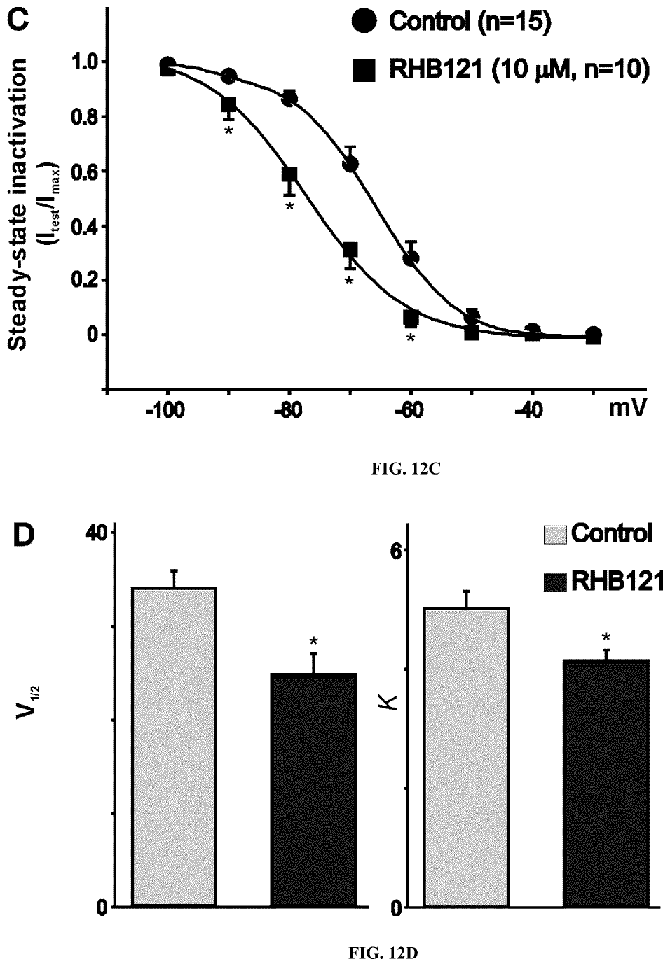

The action of N-benzamide enaminone analogs with various substitutions in the enaminone ring, including RHB62, RHB95, RHB121, RHB107, and ABA58 were tested. Typical transient Ca²⁺ currents generated in HEK 293-Cav3.2 cells by a 200 ms-voltage step to −20 mV from a holding potential of −100 mV are represented in FIG. 11A (control and following treatment with 50 mM RHB121). Cell capacitance and current densities in HEK 293-Cav3.2 cells following treatment with RHB62, RHB95, RHB 121, RHB107, and ABA58 were measured. There were no significant changes in cell capacitance following the treatment of HEK 293-Cav3.2 cells with RHB62, RHB95, RHB 121, RHB107, and ABA58 (FIG. 11B). However, exposure of HEK 293-Cav3.2 cells to 50 mM RHB62, RHB95, and RHB121 caused a significant reduction in T-type Ca²⁺ current density (FIG. 11C). RHB121 was the most effective compound in evoking a near 100% reduction in T-type Ca²⁺ currents. The effect of RHB121 was concentration-dependent (FIG. 11D). RHB121 does not alter the activation and inactivation time constants of T-type Ca²⁺ currents (FIG. 12A-B). However, treatment of HEK 293-Cav3.2 cells with 10 mM RHB121 caused a leftward shift in the steady-state inactivation of T-type Ca²⁺ currents (FIG. 12C). The voltage dependence of the steady-state inactivation was obtained by the best fit with a Boltzmann equation to assess the inactivation parameters V_{1/2} and k (FIG. 12D).

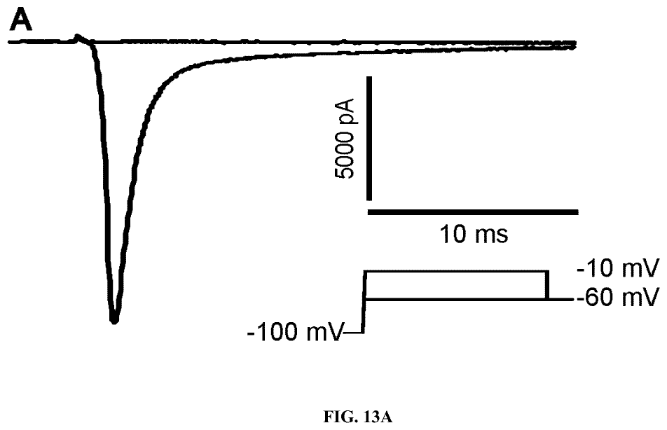

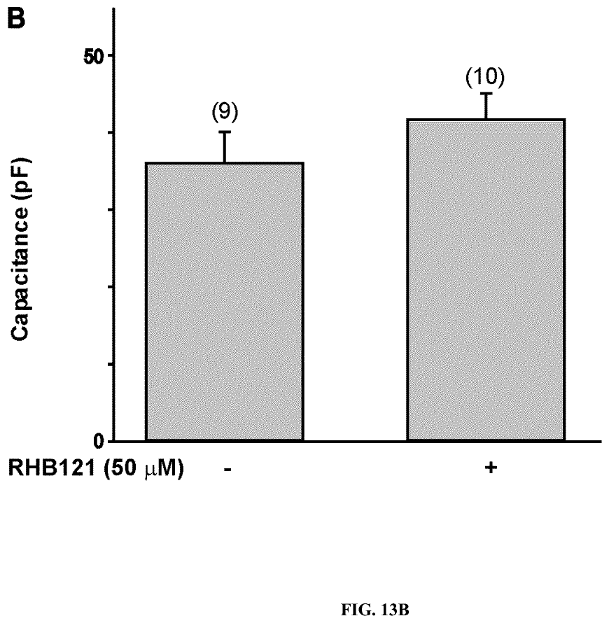

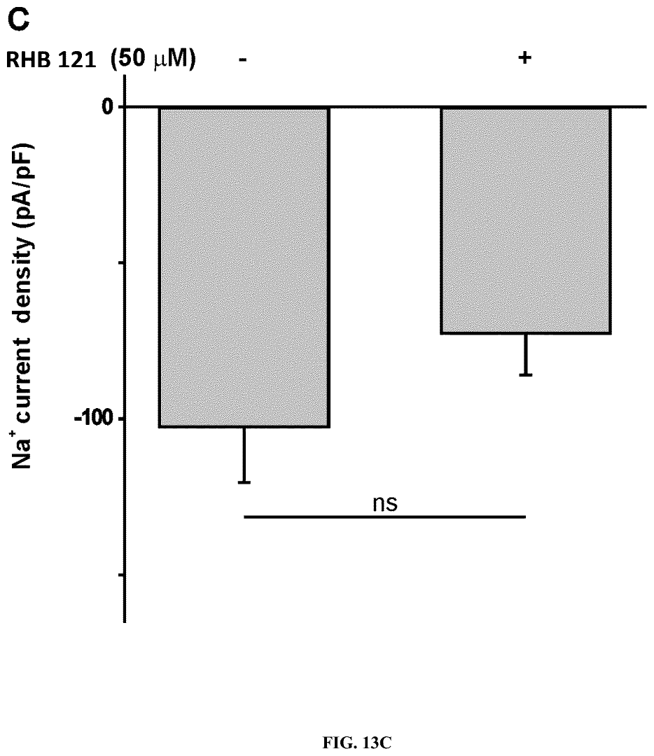

To assess the effect of RHB121 on voltage-gated sodium channels, whole-cell recordings in differentiated ND7/23 cells was performed. A typical sodium current generated in a differentiated ND7/23 cell is represented in FIG. 13A. Treatment of differentiated ND7/23 cells with 50 μM of RHB121 does not affect the call capacitance or sodium current densities compared to non-treated cells (FIG. 13. B-C).

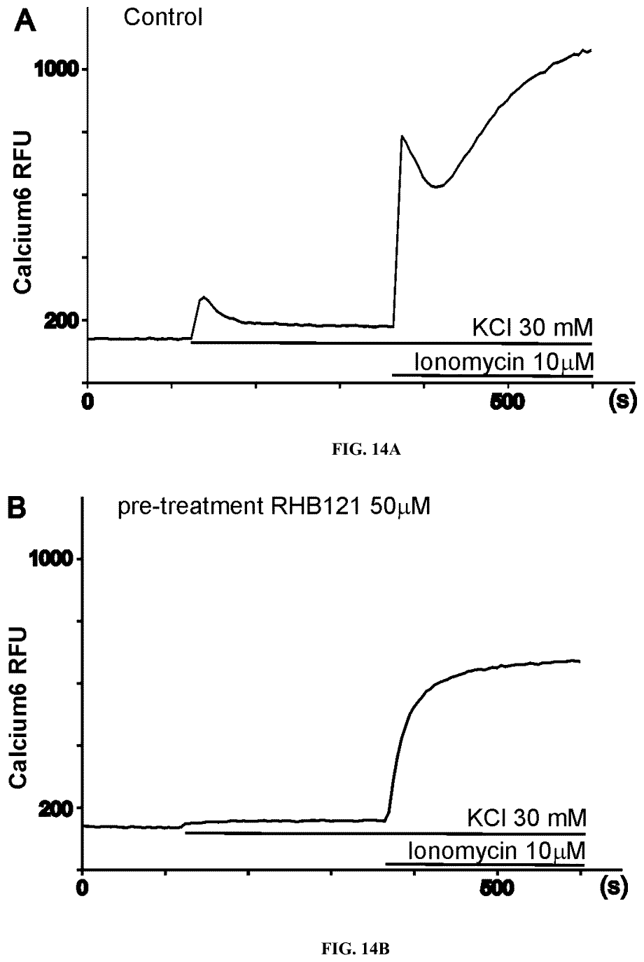

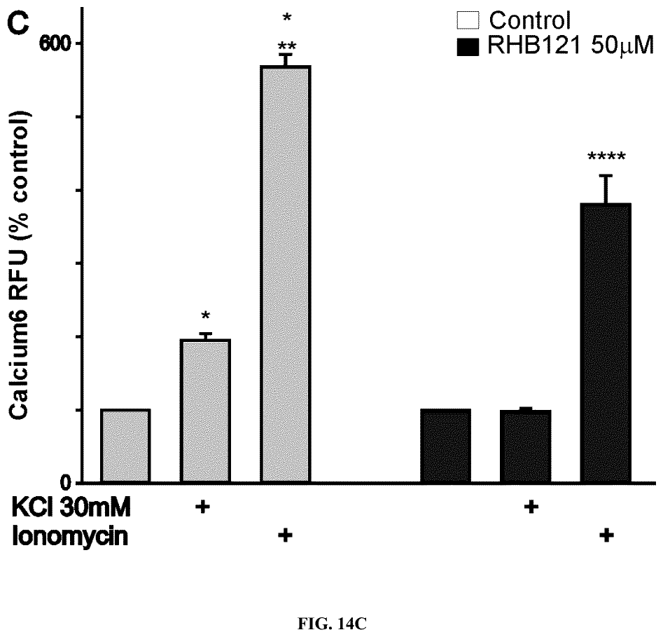

Activation of T-type Ca²⁺ channels can generate a significant increase in intracellular calcium signals. To assess the effect of RHB121 on intracellular calcium signals, differentiated ND7/23 cells with a high potassium concentration (30 mM KCl) were simulated. The effect of the calcium ionophore Ionomycin, which also can increase intracellular calcium by a different mechanism, independently of T-type Ca²⁺ channel activation, was also tested. Stimulation of differentiated ND7/23 with 30 mM KCl external solution caused a small increase in the intracellular calcium signal (FIG. 14A). Ionomycin also evokes a significant increase in intracellular calcium signal. However, treatment of differentiated ND7/23 with 50 μM RHB121 caused a complete inhibition of intracellular calcium signal evoked with 30 mM KCL solution, whereas Ionomycin still evoked a significant response in the presence of 50 uM RHB121 (FIG. 14B). These findings are summarized in FIG. 14C.

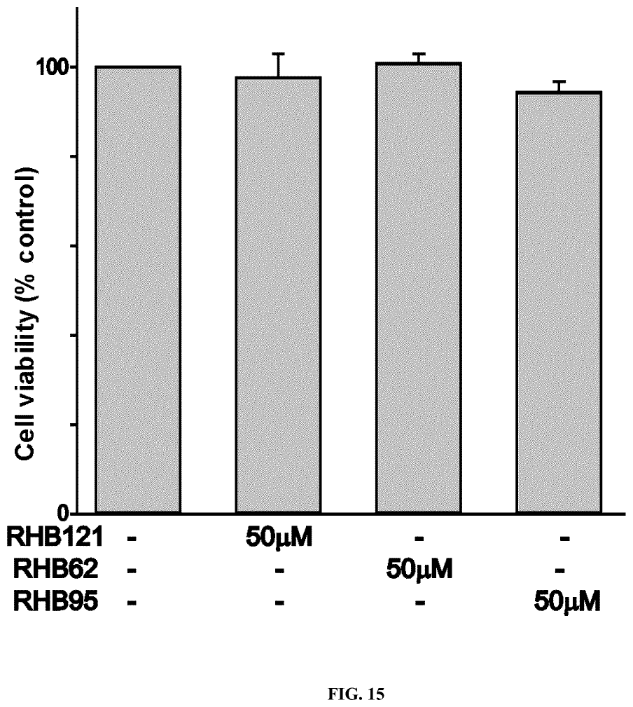

To assess the effect of RHB62, RHB95, and RHB121 on cell viability, the XTT assay was used. This assay was performed on prostate cancer cells that do not express any ion channels to test whether these compounds can alter cell viability (FIG. 15). The results indicate that RHB62, RHB95, and RHB121 do not affect the cell viability of prostate cancer cells, indicating that these compounds do not have a cytotoxic effect.

### REFERENCES

Amaye I J, Heinbockel T, Woods J, Wang Z, Martin-Caraballo M, Jackson-Ayotunde P 2018. 6 Hz Active Anticonvulsant Fluorinated N-Benzamide Enaminones and Their Inhibitory Neuronal Activity. Int J Environ Res Public Health. 15(8):1784.
Salome C, Salome-Grosjean E, Park K, Marieux P, Swendiman E D, Stables J, Kohn H 2010. Synthesis and anticonvulsant activities of (R)—N-(4′-substituted)benzyl 2-acetamido-3-methoxypropionamides. J Med Chem 53(3):1288-1305.
Wang Z, Sun L, Jackson P L, Scott R K, Heinbockel T 2011. A Substituted Anilino Enaminone Acts as a Novel Positive Allosteric Modulator of GABAA Receptors in the Mouse Brian. Journal of Pharmacology and Experimental Therapeutics 336(3):916-924.
Ghosh S, Sinha J K, Khan T, Devaraju K S, Singh P, Vaibhav K, Gaur P 2021. Pharmacological and Therapeutic Approaches in the Treatment of Epilepsy. Biomedicines. 9(5):470.
Choi H, Hayat M J, Zhang R, Hirsch L J, Bazil C W, Mendiratta A, Kato K, Javed A, Legge A W, Buchsbaum R 2016. Drug-resistant epilepsy in adults: Outcome trajectories after failure of two medications. Epilepsia 57:1152-1160.
Löscher W, Potschka H, Sisodiya S M, Vezzani A 2020. Drug Resistance in Epilepsy: Clinical Impact, Potential Mechanisms, and New Innovative Treatment Options. Pharmacol Rev. 72(3): 606-638.
Zamponi G W, Lory P, Perez-Reyes E 2010. Role of voltage-gated calcium channels in epilepsy. Pflugers Arch. 460(2):395-403.
Perez-Reyes E 2003. Molecular physiology of low-voltage-activated t-type calcium channels. Physiol Rev. 83(1):117-161.
McRory J E, Santi C M, Hamming K S, Mezeyova J, Sutton K G, Baillie D L, Stea A, Snutch T P 2001. Molecular and functional characterization of a family of rat brain T-type calcium channels. J Biol Chem. 276(6):3999-4011.
Cain S M, Snutch T P 2010. Contributions of T-type calcium channel isoforms to neuronal firing. Channels (Austin). 4(6):475-482.
Fernandez F R, Iftinca M C, Zamponi G W, Turner R W 2021. Modeling temperature- and Cav3 subtype-dependent alterations in T-type calcium channel mediated burst firing. Mol Brain. 2021 Jul. 17; 14(1):115.
Su H, Sochivko D, Becker A, Chen J, Jiang Y, Yaari Y, Beck H 2002. Upregulation of a T-type Ca2+ channel causes a long-lasting modification of neuronal firing mode after status epilepticus. J Neurosci. 22(9):3645-3655.
Chen Y, Lu J, Pan H, Zhang Y, Wu H, Xu K, Liu X, Jiang Y, Bao X, Yao Z, Ding K, Lo W H, Qiang B, Chan P, Shen Y, Wu X 2003. Association between genetic variation of CACNA1H and childhood absence epilepsy. Ann Neurol. 54(2):239-243.
Vitko I, Chen Y, Arias J M, Shen Y, Wu X R, Perez-Reyes E 2005. Functional characterization and neuronal modeling of the effects of childhood absence epilepsy variants of CACNA1H, a T-type calcium channel. J Neurosci. 25(19):4844-4855.
Edafiogho I O, Hinko C N, Chang H, Moore J A, Mulzac D, Nicholson J M, Scott K R 1992. Synthesis and anticonvulsant activity of enaminones. J Med Chem. 35(15):2798-2805.
Alexander S M, Harkless J, Butcher J R, Scott K R, Jackson-Ayotunde L P 2013. Enaminones 11. An Examination of Some Ethyl Ester Enaminone Derivatives as Anticonvulsant Agents. Bioorganic and Medicinal Chemistry 21:3272-3279.
Amaye I, Haywood R, Mandzo E, Wirick J, Jackson-Ayotunde P L 2021. Enaminones as building blocks in drug development: Recent advances. Tetrahedron 83:131984.
Wood J N, Bevan S J, Coote P R, Dunn P M, Harmar A, Hogan P, Latchman D S, Morrison C, Rougon G, Theveniau M, Wheatley S (1990) Novel cell lines display properties of nociceptive sensory neurons. Proc Biol Sci. 241(1302):187-194.
Zhang Q J, Hsia S C, Martin-Caraballo M 2017. Regulation of T-type Ca² channel expression by herpes simplex virus-1 infection in sensory-like ND7 cells. J. Neurovirol. 23(5):657-670.
Wolfe J T, Wang H, Perez-Reyes E, Barrett P Q 2002. Stimulation of recombinant Ca(v)3.2, T-type, Ca (2+) channel currents by CaMKIIgamma(C). J Physiol. 538(Pt 2):343-55.
Dey D, Shepherd A, Pachuau J, Martin-Caraballo M 2011. Leukemia inhibitory factor regulates trafficking of T-type Ca²⁺ channels. Am J Physiol Cell Physiol. 300(3):C576-587.
Klöckner U, Lee J H, Cribbs L L, Daud A, Hescheler J, Pereverzev A, Perez-Reyes E, Schneider T 1999. Comparison of the Ca²⁺ currents induced by expression of three cloned alpha1 subunits, alpha1G, alpha1H and alpha1I, of low-voltage-activated T-type Ca²⁺ channels Eur J Neurosci. 11(12):4171-4178.
Fang Y, Kirkland J, Amaye I J, Jackson-Ayotunde P, George M (Jr) 2019. Molecular Docking Studies on Anticonvulsant Enaminones Inhibiting Voltage-Gated Sodium Channels. Open J Phys Chem. 9(4):241-257.
Yin K, Baillie G J, Vetter 12016. Neuronal cell lines as model dorsal root ganglion neurons: A transcriptomic comparison. Mol Pain 12:1744806916646111.
Santi C M, Cayabyab F S, Sutton K G, McRory J E, Mezeyova J, Hamming K S, Parker D, Stea A, Snutch T P 2002. Differential inhibition of T-type calcium channels by neuroleptics. J Neurosci. 22(2):396-403.
Orestes P, Bojadzic D, Chow R M, Todorovic S M 2009. Mechanisms and functional significance of inhibition of neuronal T-type calcium channels by isoflurane. Mol Pharmacol. 75(3):542-554.
Strege P R, Sha L, Beyder A, Bernard C E, Perez-Reyes E, Evangelista S, Gibbons S J, Szurszewski J H, Farrugia G 2010. T-type Ca(2+) channel modulation by otilonium bromide. Am J Physiol Gastrointest Liver Physiol. 298(5):G706-713.
Murbartiin J, Arias J M, Lee J H, Gomora J C, Perez-Reyes E 2002. Alternative splicing of the rat Ca(v)3.3 T-type calcium channel gene produces variants with distinct functional properties. FEBS Lett. 528(1-3):272-278.
Talley E M, Cribbs L L, Lee J H, Daud A, Perez-Reyes E, Bayliss D A 1999. Differential distribution of three members of a gene family encoding low voltage-activated (T-type) calcium channels. J Neurosci. 19(6):1895-1911.
Yunker A M, Sharp A H, Sundarraj S, Ranganathan V, Copeland T D, McEnery M W 2003. Immunological characterization of T-type voltage-dependent calcium channel CaV3.1 (alpha 1G) and CaV3.3 (alpha 1I) isoforms reveal differences in their localization, expression, and neural development. Neuroscience. 117(2):321-335.
Aguado C, Garcia-Madrona S, Gil-Minguez M, Lujin R 2016. Ontogenic Changes and Differential Localization of T-type Ca(2+) Channel Subunits Cav3.1 and Cav3.2 in Mouse Hippocampus and Cerebellum. Front Neuroanat. 10:83.
Joksovic P M, Bayliss D A, Todorovic S M 2005. Different kinetic properties of two T-type Ca2+ currents of rat reticular thalamic neurones and their modulation by enflurane. J Physiol. 566(Pt 1):125-142.
Cribbs L 2010. T-type calcium channel expression and function in the diseased heart. Channels (Austin). 4(6):447-452.
Nelson M T, Todorovic S M, Perez-Reyes E. The role of T-type calcium channels in epilepsy and pain. Curr Pharm Des. 2006; 12(18):2189-97.
Strege P R, Bernard C E, Ou Y, Gibbons S J, Farrugia G 2005. Effect of mibefradil on sodium and calcium currents. Am J Physiol Gastrointest Liver Physiol. 289(2):G249-253.
Sills G J, Rogawski M A 2020. Mechanisms of action of currently used antiseizure drugs. Neuropharmacology 168:107966.
Leresche N, Parri H R, Erdemli G, Guyon A, Turner J P, Williams S R, Asprodini E, Crunelli V 1998. On the action of the anti-absence drug ethosuximide in the rat and cat thalamus. J Neurosci. 18(13):4842-4853.
Nam G 2018. T-type calcium channel blockers: a patent review (2012-2018). Expert Opin Ther Pat. 28(12):883-901.
Tringham E, Powell K L, Cain S M, Kuplast K, Mezeyova J, Weerapura M, Eduljee C, Jiang X, Smith P, Morrison J L, Jones N C, Braine E, Rind G, Fee-Maki M, Parker D, Pajouhesh H, Parmar M, O'Brien T J, Snutch T P 2012. T-type calcium channel blockers that attenuate thalamic burst firing and suppress absence seizures. Sci Transl Med. 4(121):121ra19.
McArthur J R, Motin L, Gleeson E C, Spiller S, Lewis R J, Duggan P J, Tuck K L, Adams D J. Inhibition of human N- and T-type calcium channels by an ortho-phenoxyanilide derivative, MONIRO-1. Br J Pharmacol. 2018 June; 175(12):2284-2295.

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

We claim:

1. A method of treating a disease, condition, or disorder, said method comprising contacting one or more cells of a subject with one or more of the compounds of the Formula I or a pharmaceutically acceptable salt thereof: 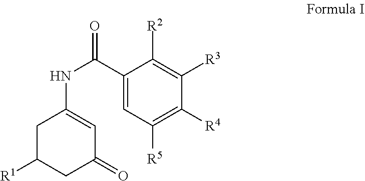
wherein
R¹ is C₁-C₆-alkyl or C₁-C₆-trifluoroalkyl; and
each of R², R³, R⁴, or R⁵ is independently chosen from H, halide, alkoxy, C₁-C₆-trifluoroalkyl, fluorinated alkoxy, fluorinated thio group, CN, and NO₂.

2. The method of claim 1, wherein said compound of Formula I is:

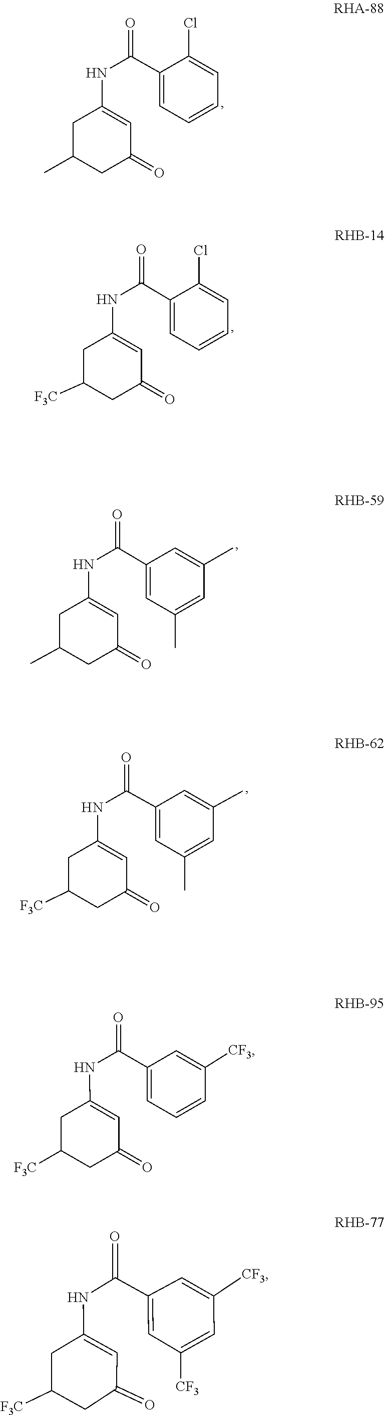

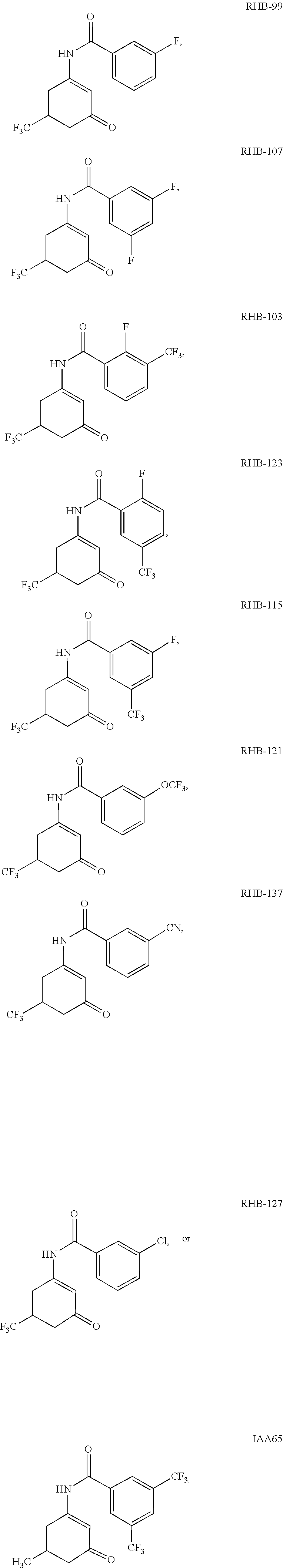

3. The method of claim 1, wherein said compound of Formula I is: 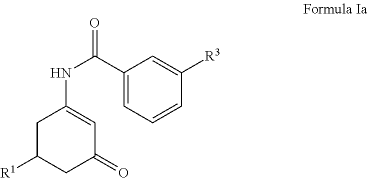
wherein each of R², R⁴, and R⁵ is H.

4. The method of claim 1, wherein said disease, condition and/or disorder involves the modulating (e.g. inhibition) of one or more calcium channels and/or one or more sodium channels.

5. The method of claim 4, wherein said calcium channel is T-type.

6. The method of claim 5, wherein said calcium channel is Cav3.2 and/or Cav3.3 subtype.

7. The method of claim 1, wherein said disease, condition and/or disorder is a neurological disease, condition or disorder, pain disease, condition or disorder, sleep disease, condition or disorder, and/or anxiety disease, condition or disorder.

8. The method of claim 1, wherein said disease, condition and/or disorder is a seizure disorder.

9. The method of claim 2, wherein the compound is chosen from:

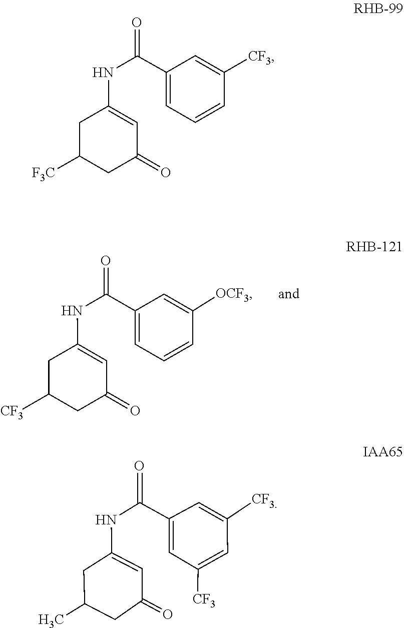 [SMILES: O=C1C=C(NC(=O)c2cccc(C(F)(F)F)c2)CC(C(F)(F)F)C1 ; O=C1C=C(NC(=O)c2cccc(OC(F)(F)F)c2)CC(C(F)(F)F)C1 ; CC1CC(=O)C=C(NC(=O)c2cc(C(F)(F)F)cc(C(F)(F)F)c2)C1]

10. The method of claim 9, wherein the compound is:

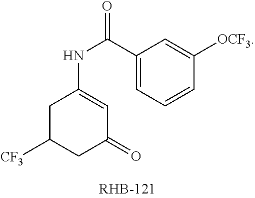 [SMILES: O=C1C=C(NC(=O)c2cccc(OC(F)(F)F)c2)CC(C(F)(F)F)C1]

11. The method of claim 9, wherein the compound is:

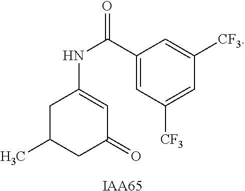

12. The method of claim 1, wherein each of R¹ and R³ is C₁-C₆-trifluoroalkyl.

13. The method of claim 1, wherein each of R¹ and R³ is —CF₃.

14. The method of claim 1, wherein R¹ is C₁-C₆-trifluoroalkyl and R³ is fluorinated alkoxy.

15. The method of claim 1, wherein R¹ is —CF₃ and R³ is —OCF₃.
